(19) United States
(12) Patent Application Publication
(10) Pub. No.: US 2023/0227828 A1
(43) Pub. Date: Jul. 20, 2023

(54) COMPOSITION FOR PREVENTING OR TREATING ALZHEIMER'S DISEASE COMPRISING INHIBITOR OF ATLASTIN 2, AND METHOD FOR DIAGNOSING ALZHEIMER'S DISEASE BY DETERMINING ATLASTIN 2

(71) Applicant: Research & Business Foundation Sungkyunkwan University, Suwon-si (KR)

(72) Inventors: Dong Gyu JO, Suwon-si (KR); Ji Hoon HAN, Suwon-si (KR)

(73) Assignee: Research & Business Foundation Sungkyunkwan University, Suwon-si (KR)

(21) Appl. No.: 18/146,818

(22) Filed: Dec. 27, 2022

(30) Foreign Application Priority Data
Dec. 27, 2021 (KR) 10-2021-0188902

(51) Int. Cl. C12N 15/113; A61K 31/7088; G01N 33/68

(52) U.S. Cl. CPC C12N 15/113; A61K 31/7088; G01N 33/6896; C12N 2310/141; C12Q 2600/158

(57) ABSTRACT

The present invention relates to a composition for preventing or treating Alzheimer’s disease, containing an inhibitor of ATL2, and a method of diagnosing the disease based on the measurement of the ATL2. In the present invention, it was found that PS1 mutants may result in mitochondrial dysfunction, such as increased binding between endoplasmic reticulum and mitochondria, increased mitochondrial ROS production, decreased mitochondrial membrane potential, decreased ATP production, decreased complex I activity, and decreased peroxidase activity, in brain glioma cells and that the PS1 mutants may abnormally increase the binding between endoplasmic reticulum and mitochondria by elevating the expression of the ATL2 in the brain. In addition, when the ATL2 was knocked down, it was observed that the binding between endoplasmic reticulum and mitochondria was lowered and that the expression of the ATL2 was elevated in the brains of Alzheimer’s disease animal models and patients. Accordingly, it is expected that it may possible to effectively prevent or treat Alzheimer’s disease by inhibiting the expression or activity of the ATL2 and that it may possible to diagnose the disease, predict the risk of developing the disease, and screen therapeutic agents for the disease, by measuring the level of the expression or activity of the ATL2.

### CROSS-REFERENCE TO RELATED APPLICATION

This application claims priority to and the benefit of Korean Patent Application No. 10-2021-0188902, filed on Dec. 27, 2021, the invention of which is incorporated herein by reference in its entirety.

### REFERENCE TO AN ELECTRONIC SEQUENCE LISTING

The contents of the electronic sequence listing (“NewApp_0421350059_SequenceListing.xml”; Size is 21 KB and it was created on Dec. 27, 2022) is herein incorporated by reference in its entirety.

### BACKGROUND

### 1. Field of the Invention

The present invention relates to a composition for preventing or treating Alzheimer’s disease comprising an inhibitor of Atlastin 2, a method of diagnosing Alzheimer’s disease by determining the Atlastin 2, etc.

### 2. Discussion of Related Art

Alzheimer’s disease patients account for the largest proportion of dementia patients, and the disease is a degenerative brain disease for which no effective treatment has been found yet. Alzheimer’s disease is characterized by accumulation of amyloid-beta (Aβ) and tau proteins in the brain, chronic neuroinflammation, and mitochondrial dysfunction. Previous studies on Alzheimer’s disease have focused on the accumulation of the amyloid beta and tau proteins, but most of the development of new drugs targeting these two substances has failed. Accordingly, there is a demand for a therapeutic agent targeting the pathological phenotype of Alzheimer’s disease in addition to the two substances, and extensive research is being conducted to overcome Alzheimer’s disease with a multifaceted approach.

Aging is a major risk factor for developing Alzheimer’s disease, and it leads to progressive decline in physiological function eventually resulting in a range of pathological conditions such as cancer, arthritis, stroke, and neurodegenerative diseases and causes irreversible cell damage and aging-related degenerative diseases due to oxygen radicals (reactive oxygen species: ROS) generated during normal metabolic processes.

Mitochondria are considered a major source and target of free radicals, and the accumulation of damaged mitochondria may impair respiratory chain function and increase the production of the ROS. It has also been reported that progressive damage to the mitochondrial membrane by free radicals may lead to increased superoxide production and an age-related decrease in the production of functionally competent mitochondria and cellular ATP. In relation to aging, the role of mitochondria has been further emphasized as more and more evidence has emerged that the accumulation of somatic mutants in mitochondrial DNA (mtDNA) contributes significantly to aging and degenerative diseases in humans.

Since aging is a major risk factor for developing Alzheimer’s disease, it is reasonable to suggest that mitochondrial dysfunction affects the progression of the disease. Indeed, previous studies have shown that mitochondrial dysfunction and oxidative stress play an important role in the early pathogenesis of Alzheimer’s disease. In addition, it has been reported that oxidative damage occurs even before an Aβ plaque is formed, supporting that mitochondrial dysfunction and oxidative stress may result in Alzheimer’s disease. The amyloid cascade hypothesis that the primary event of Alzheimer’s disease neurodegeneration is the production of Aβ is the most convincing hypothesis for familial Alzheimer’s disease (FAD), but not for sporadic Alzheimer’s disease (SAD), so the mitochondrial cascade hypothesis was proposed in 2004. This theory suggests that mitochondrial dysfunction represents the major pathology of the SAD and leads to the formation of Aβ plaques and neurofibrillary tangles. The theory has been supported because various forms of mitochondrial dysfunction have been reported in connection with Alzheimer’s disease: abnormal mitochondrial morphology, inhibition of oxidative phosphorylation, increased production of ROS, endoplasmic reticulum (ER)-mitochondria membrane (MAM), damaged mitochondrial biogenesis, etc.

Meanwhile, an aspartyl protease, Presenilin-1 (PS1), is a catalytic subunit of γ-secretase and mediates the cleavage of type I transmembrane proteins, including an amyloid precursor protein (APP), in the transmembrane area. Sequential cleavage of the APP by beta-secretase and γ-secretase leads to the production of Aβ, which is deposited as plaques in the brains of Alzheimer’s disease patients. To date, 300 or more mutants have been identified within the entire sequence of PS1, most of which are associated with early onset of the FAD. These FAD-linked PS1 mutants induce consistent changes in PS1 conformation, leading to a shift in the Aβ42/40 ratio. Moreover, the conformation of endogenous PS1 observed in people undergoing normal aging and in patients with the SAD is altered to a “closed” conformation similar to that observed in the FAD-linked PS1 mutants. Thus, the PS1 plays an important role in the pathogenesis of the FAD and the SAD. The PS1 is known to be localized to numerous compartments of cells, including endoplasmic reticulum, Golgi apparatus, nuclear envelope, endosomes, lysosomes, plasma membrane, and mitochondria. More specifically, it was reported in 2009 that the PS1 is highly abundant in the MAM, a subdomain of endoplasmic reticulum in contact with mitochondria, and the PS1 is known to play an important role in phospholipid biosynthesis, cholesterol esterification, calcium transport, and mitochondrial and endoplasmic reticulum homeostasis.

Accordingly, the inventors of the present invention sought to suggest the possibility of developing a therapeutic agent for Alzheimer’s disease by investigating the effect and mechanism of the PS1 mutants on mitochondrial function in relation to Alzheimer’s disease.

### SUMMARY

The inventors investigated the effects of five different PS1 mutants (A431E, E280A, H163R, M146V, and Δexon9) identified in relation to Alzheimer’s disease on mitochondrial function, and it was observed that the PS1 mutants resulted in increased binding between endoplasmic reticulum and mitochondria, increased production of mitochondrial ROS, decreased mitochondrial membrane potential, decreased production of ATP, decreased complex I activity, decreased peroxidase activity, etc. in brain glioma cells. Furthermore, it was found that the PS1 mutants abnormally increased the binding between endoplasmic reticulum and mitochondria by increasing the expression of Atlastin 2 (ATL2) in the brain. In addition, it was observed that the expression of the ATL2 was elevated in the brains of Alzheimer’s disease animal models and patients, and the present invention was completed based on such findings.

Therefore, the purpose of the present invention is to provide a pharmaceutical composition for preventing or treating Alzheimer’s disease, comprising an inhibitor of the expression or activity of the Atlastin 2 (ATL2) gene or protein as an active ingredient.

Another purpose of the present invention is to provide a composition for diagnosing Alzheimer’s disease, comprising, as an active ingredient, an agent for measuring the level of the expression or activity of the Atlastin 2 (ATL2) gene or protein and a kit comprising the composition.

Still another purpose of the present invention is to provide a method of providing information for diagnosing Alzheimer’s disease or predicting the risk of developing the disease, comprising measuring the level of the expression or activity of the Atlastin 2 (ATL2) gene or protein.

A further purpose of the present invention is to provide a method of screening candidate substances for treatment of Alzheimer’s disease based on the measurement of the level of the expression or activity of the Atlastin 2 (ATL2) gene or protein.

However, the purposes of the present invention are not limited to the above-mentioned purposes, and other purposes not mentioned above are to be clearly understood through the following description by a person having ordinary skill in the technical field to which the present invention belongs.

In order to achieve the aforementioned purposes, the present invention provides the pharmaceutical composition for preventing or treating Alzheimer’s disease, comprising the inhibitor of the expression or activity of the Atlastin 2 (ATL2) gene or protein as an active ingredient.

According to an embodiment of the present invention, the inhibitor of the expression or activity of the ATL2 gene may be one or more selected from the group consisting of microRNA (miRNA), small interference RNA (siRNA), short hairpin RNA (shRNA), peptide nucleic acid (PNA), antisense nucleotides, and ribozymes, which specifically bind to the mRNA of the ATL2 gene, but is not limited thereto.

According to an embodiment of the present invention, the inhibitor of the expression or activity of the ATL2 protein may be one or more selected from the group consisting of compounds, peptides, peptide mimics, substrate analogs, aptamers, and antibodies, which specifically bind to the ATL2 protein, but is not limited thereto.

According to an embodiment of the present invention, the inhibitor of the expression or activity of the ATL2 gene or protein may lower the binding between endoplasmic reticulum (ER) and mitochondria in the brain, but is not limited thereto.

According to an embodiment of the present invention, the inhibitor of the expression or activity of the ATL2 gene or protein may inhibit the production of mitochondrial superoxide, but is not limited thereto.

In addition, the present invention provides a composition for diagnosing Alzheimer’s disease, comprising, as an active ingredient, an agent for measuring the level of the expression or activity of the Atlastin 2 (ATL2) gene or protein.

The present invention provides a kit for diagnosing Alzheimer’s disease, comprising the composition for diagnosing the disease.

According to another embodiment of the present invention, an agent for measuring the level of the expression or activity of the ATL2 gene may be one or more selected from the group consisting of primers, probes, and antisense nucleotides, which specifically bind to the mRNA of the ATL2 gene, but is not limited thereto.

According to another embodiment of the present invention, the agent for measuring the level of the expression or activity of the ATL2 protein may be antibodies or aptamers that specifically bind to the ATL2 protein, but is not limited thereto.

In addition, the present invention provides a method of providing information for diagnosing Alzheimer’s disease, involving measuring the level of the expression or activity of the Atlastin 2 (ATL2) gene or protein in a sample collected from the brain of a subject.

According to still another embodiment of the present invention, the method may further comprise diagnosing a subject with Alzheimer’s disease when the level of the expression or activity of the ATL2 gene or protein is higher than that of a normal control group, but is not limited thereto.

In addition, the present invention provides a method of providing information for predicting the risk of developing Alzheimer’s disease, comprising measuring the level of the expression or activity of the Atlastin 2 (ATL2) gene or protein in a sample collected from the brain of a subject.

According to yet another embodiment of the present invention, the method may further comprise predicting a high risk of developing Alzheimer’s disease when the level of the expression or activity of the ATL2 gene or protein is higher than that of a normal control group, but is not limited thereto.

Furthermore, the present invention provides a method of screening candidate substances for treatment of Alzheimer’s disease, which comprises the following steps:
treating presenilin-1 (PS1)-overexpressed cells collected from a subject having the Alzheimer’s disease with a candidate substance;
measuring the level of the expression or activity of the ATL2 gene or protein in the cells; and
selecting a candidate substance as a candidate substance for treatment of Alzheimer’s disease when the level of the expression or activity of the ATL2 gene or protein is lower than the level measured before the cells were treated with the candidate substance.

According to an embodiment of the present invention, the ATL2 protein may comprise an amino acid sequence of SEQ ID NO: 1, but is not limited thereto.

According to an embodiment of the present invention, the ATL2 gene may comprise a base sequence encoding the amino acid sequence of SEQ ID NO: 1, but is not limited thereto.

In addition, the present invention provides a method of treating or alleviating Alzheimer’s disease, comprising administering the composition comprising, as an active ingredient, the inhibitor of the expression or activity of the Atlastin 2 (ATL2) gene or protein to a subject in need thereof.

In addition, the present invention provides a use of the composition comprising, as an active ingredient, the inhibitor of the expression or activity of the Atlastin 2 (ATL2) gene or protein for preventing, treating or alleviating Alzheimer’s disease.

In addition, the present invention provides a use of the composition comprising, as an active ingredient, the inhibitor of the expression or activity of the Atlastin 2 (ATL2) gene or protein for preparing drugs for preventing, treating, or alleviating Alzheimer’s disease.

In addition, the present invention provides a method of diagnosing Alzheimer’s disease or predicting the risk of developing the disease, comprising measuring the level of the expression or activity of the Atlastin 2 (ATL2) gene or protein in a sample collected from the brain of a subject.

In addition, the present invention provides a use of the composition comprising, as an active ingredient, the agent for measuring the level of the expression or activity of the Atlastin 2 (ATL2) gene or protein for diagnosing Alzheimer’s disease.

In addition, the present invention provides a use of the composition comprising, as an active ingredient, the agent for measuring the level of the expression or activity of the Atlastin 2 (ATL2) gene or protein for preparing an agent for diagnosing Alzheimer’s disease.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1A is a schematic diagram showing a system for inducing PS1 mutants by tetracycline according to an embodiment of the present invention;

FIGS. 1B and 1C are views showing the results of performing the western blot on PS1 mutants for PS1 in H4^{PS1} cell lines before and after treatment with tetracycline according to an embodiment of the present invention;

FIGS. 1D and 1E are views showing the results of performing the western blot on PS1 mutants for APP-CTF in H4^{PS1} cell lines before and after treatment with tetracycline according to an embodiment of the present invention;

FIG. 2A is a view showing the images of H4^{PS1} cell lines viewed with a confocal microscope before and after treatment with tetracycline according to an embodiment of the present invention;

FIG. 2B is a view showing the results of quantifying the length of mitochondria in H4^{PS1} cell lines before and after treatment with tetracycline according to an embodiment of the present invention;

FIG. 2C shows the results of observing the distribution of mitochondria in the H4^{PS1} cell lines using z-stack imaging of a confocal microscope according to an embodiment of the present invention;

FIG. 2D is a view showing the results of the western blot on PS1 mutants for proteins (OPA1, MFN2, and DRP1) related to mitochondrial dynamics in H4^{PS1} cell lines and the quantification of the results according to an embodiment of the present invention;

FIG. 3A is a view showing the confocal images of H4^{PS1} cells labeled with MitoTracker (red) and ERTracker (green), taken before and after treatment with tetracycline, and the results of analyzing the images by the line scan according to an embodiment of the present invention;

FIG. 3B is a view showing weighted colocalization coefficients between mitochondria and endoplasmic reticulum in H4^{PS1} cells observed before and after treatment with tetracycline according to an embodiment of the present invention;

FIG. 3C is a view showing the images of H4^{PS1} cell lines viewed with a transmission electron microscope before and after treatment with tetracycline according to an embodiment of the present invention;

FIG. 3D is a view showing the results of quantifying mitochondria in contact with endoplasmic reticulum in H4^{PS1} cell lines before and after treatment with tetracycline according to an embodiment of the present invention;

FIG. 4A is a view showing the results of quantifying ROS production measured in H4^{PS1} cell lines before and after treatment with tetracycline according to an embodiment of the present invention;

FIG. 4B is a view showing the confocal images of H4^{PS1} cell lines stained with MitoSOX (red) and Hoechst (blue), taken before and after treatment with tetracycline, according to an embodiment of the present invention;

FIG. 4C is a view showing the results of quantifying the production of mitochondrial superoxide in H4^{PS1} cell lines before and after treatment with tetracycline according to an embodiment of the present invention;

FIG. 4D is a view showing the results of quantifying the activity of complex I of mitochondria in H4^{PS1} cell lines by using the assay for the activity of complex I enzyme before and after treatment with tetracycline according to an embodiment of the present invention;

FIG. 4E is a view showing the results of quantifying peroxidase activity in H4^{PS1} cell lines before and after treatment with tetracycline according to an embodiment of the present invention;

FIG. 4F is a view showing the results of quantifying the membrane potential of mitochondria in H4^{PS1} cell lines before and after treatment with tetracycline according to an embodiment of the present invention;

FIG. 4G is a view showing the results of quantifying the level of total ATP in H4^{PS1} cell lines before and after treatment with tetracycline according to an embodiment of the present invention;

FIG. 4H is a view showing the results of observing mitochondrial functions in H4^{PS1} cell lines before and after treatment with tetracycline according to an embodiment of the present invention;

FIG. 5 is a view showing the results of analyzing hippocampal oxygen consumption by observing basal respiration, ATP-linked respiration, and proton leak in H4^{PS1} cell lines before and after treatment with tetracycline according to an embodiment of the present invention;

FIGS. 6A to 6E relate to the expression profile of hippocampal genes of PS1M146V knock-in mice according to an embodiment of the present invention. FIG. 6A is a view showing hierarchical clustering, FIG. 6B is a view showing a volcano plot of DEGs, FIG. 6C is a view showing the number of differentially upregulated and downregulated counts by z-ratio and P-value, FIG. 6D is a view showing a list of DEGs related to mitochondria or endoplasmic reticulum, and FIG. 6E is a view showing the results of an analysis of gene ontology and pathway frequency of DEGs;

FIG. 6F is a view showing the results of an analysis of major components of the hippocampus of wild-type and PS1M146V knock-in mice according to an embodiment of the present invention;

FIG. 7A is a view showing the results of measuring the mRNA expression levels of ATL1, ATL2, and ATL3 in H4^{PS1} cell lines before and after treatment with tetracycline according to an embodiment of the present invention;

FIG. 7B is a view showing the results of performing the western blot on ATL1, ATL2, and ATL3 in the H4^{PS1A431E} and H4^{PS1M146V} cell lines before and after treatment with tetracycline according to an embodiment of the present invention;

FIG. 7C is a view showing the results of performing the western blot on ATL2 in the H4^{PS1M146V} cell line before and after treatment with tetracycline or transfection with siRNA according to an embodiment of the present invention;

FIG. 7D is a view showing the confocal images of the H4^{PS1M146V} cell lines labeled with Mito-Tracker (red) and ER-Tracker (green) before and after treatment with tetracycline or transfection with siRNA according to an embodiment of the present invention;

FIG. 7E is a view showing the results of observing weighted colocalization coefficients between mitochondria and endoplasmic reticulum in the H4^{PS1M146V} cell line before and after treatment with tetracycline or transfection with siRNA according to an embodiment of the present invention;

FIG. 7F is a view showing the results of quantifying the production of mitochondrial superoxide in the H4^{PS1M146V} cell line before and after treatment with tetracycline or transfection with siRNA according to an embodiment of the present invention;

FIG. 7G is a view showing the results of quantifying mitochondrial membrane potential in the H4^{PS1M146V} cell line before and after treatment with tetracycline or transfection with siRNA using a TMRM probe according to an embodiment of the present invention;

FIG. 8A is a view showing the results of performing the western blot on ATL1, ATL2, ATL3, and Tau proteins in the hippocampus of 7-month-old 3xTg-AD mice and wild-type mice according to an embodiment of the present invention;

FIG. 8B is a view showing the results of performing the western blot on ATL1, ATL2, ATL3, and Tau proteins in the hippocampus of 12-month-old 3xTg-AD mice and wild-type mice according to an embodiment of the present invention;

FIG. 8C is a view showing the results of performing the western blot on ATL1, ATL2, ATL3, and BACE1 in the inferior parietal lobule of Alzheimer’s disease patients and controls according to an embodiment of the present invention;

FIG. 8D is a view showing the results of observing the expression levels of ATL1, ATL2, and ATL3 genes in the frontal white matter (FWM) of Alzheimer’s disease subjects (red, n=33) and non-Alzheimer’s disease subjects (blue, n=36) according to an embodiment of the present invention; and

FIG. 9 is a schematic view showing the mechanism of the action for mitochondrial dysfunction according to the type of PS1 mutants according to an embodiment of the present invention.

### DETAILED DESCRIPTION OF EXEMPLARY EMBODIMENTS

In an experimental example of the present invention, it was observed that, when PS1 mutants were induced by tetracycline, the PS1 mutants were successfully induced by treatment with the tetracycline in H4^{PS1} cell lines so that PS1 was overexpressed (see Experimental Example 1).

In another experimental example of the present invention, the size and shape of mitochondria in each H4^{PS1} cell line mutants were observed while the PS1 mutants were induced, and it was observed that mitochondrial fragments were increased, shortened, point-like, and spherical in the H4^{PS1Δexon9} cell line and that mitochondria were relatively more aggregated in a cytoplasm. In addition, the expression levels of GTPase dynamin-related protein 1 (DRP1) and mitofusin 2 (MFN2), among proteins related to mitochondrial dynamics, significantly dropped after the PS1 mutants had been induced in the H4^{PS1Δexon9} cell line (see Experimental Example 2).

In still another experimental example of the present invention, an interaction between endoplasmic reticulum and mitochondria in each H4^{PS1} cell line was observed while PS1 mutants were induced, and it was observed that the ER-mitochondria colocalization was elevated in the H4^{PS1A431E}, H4^{PS1E280A}, H4^{PS1H163R}, and H4^{PS1M146V} cell lines and that there was a significant increase in the proportion of the mitochondria in contact with the endoplasmic reticulum (see Experimental Example 3).

In yet another experimental example of the present invention, changes in mitochondrial function were observed in each H4^{PS1} cell line while PS1 mutants were induced, and it was observed that ROS production was increased in all H4^{PS1} cell lines except for the H4^{PS1E280A} cell line; mitochondrial O_{2̇̇}˙⁻was significantly increased in the H4^{PS1A431E,} H4^{PS1E280A}, H4^{PS1M146V} and H4^{PS1Δexon9} cell lines; and the activity of complex I was significantly lowered in the H4^{PS1A431E} cell line. In addition, after overexpressed PS1 mutants were induced, significantly impaired peroxidase activity was observed in the H4^{PS1A431E} and H4^{PS1M146V} cell lines, mitochondrial membrane potential was significantly reduced in all of the H4^{PS1} mutant cell lines, and ATP levels were significantly decreased in the H4^{PS1A431E,} H4^{PS1M146V} and H4^{PS1Δexon9} cell lines (see Experimental Example 4).

In yet another experimental example of the present invention, changes in mitochondrial bioenergetics occurring as PS1 mutants were induced were observed, and it was found that, after overexpressed PS1 mutants were induced, a basal oxygen consumption rate (OCR) was significantly increased in the H4^{PS1H163R} cell line and that there was no significant difference in ATP-linked respiration and proton leak in all the H4^{PS1} cell lines (see Experimental Example 5).

In yet another experimental example of the present invention, the gene expression profile of hippocampal samples from PS1M146V knock-in mice was examined, and the examination showed that the gene expression profile of the PS1M146V knock-in mice was distinct from that of wild-type mice. Furthermore, by analyzing the main components of the hippocampus of the PS1M146V knock-in mice, it was found that there was a clear difference between the group of the wild-type mice and the group of the PS1M146V knock-in mice and that the ratio of neurons, microglia and astrocytes in the brains of the PS1M146V knock-in mice did not change (see Experimental Example 6).

In yet another experimental example of the present invention, the expression of Atlastin 2 (ATL2) in each H4^{PS1} cell line was examined while PS1 mutants were induced, and the examination showed that the expression of ATL2 mRNA and protein was significantly elevated in the H4^{PS1A431E} and H4^{PS1M146V} cell lines. In addition, the endoplasmic reticulum-mitochondria colocalization and the level of mitochondrial superoxide, which had been elevated in the H4^{PS1M146V} cell line, returned to their normal levels together with the impaired mitochondrial membrane potential upon knocking down the ATL2 (see Experimental Example 7).

In a further experimental example of the present invention, it was found that the expression level of the ATL2 gene or protein rose in the brains of Alzheimer’s disease-affected mice and patients (see Experimental Example 8).

Accordingly, according to the present invention, there may be provided a pharmaceutical composition for preventing or treating Alzheimer’s disease, comprising an inhibitor of the expression or activity of the ATL2 gene or protein as an active ingredient.

In the present invention, “Alzheimer’s disease (AD)” refers to a disease that results in the death of cerebral neurons (nerve cells) involved in learning or memory and causes people to slowly lose their memory loss and mental abilities such as computational ability, language ability, spatio-temporal understanding, and judgment. Alzheimer’s disease is the most common degenerative brain disease causing dementia, accounting for 60 to 70 percent of dementia cases. The exact mechanism for and cause of the onset of Alzheimer’s disease is not known. Currently, it is known that a key mechanism for the development of this disease is that a small protein called beta-amyloid is excessively produced and deposited in the brain and has a harmful effect on brain cells, but hyperphosphorylation, inflammatory response, oxidative damage, etc. of tau protein, which plays an important role in maintaining the skeleton of brain cells, also appear to cause damage to brain cells and contribute to the development of the disease.

In the present invention, the ATL2 protein may comprise the amino acid sequence of SEQ ID NO: 1 (NCBI Reference Sequence: NP_071769.2), but is not limited thereto.

In the present invention, the ATL2 gene as a gene encoding the ATL2 protein may comprise a base sequence encoding the amino acid sequence of SEQ ID NO: 1 and may comprise the base sequence of SEQ ID NO: 2 (NCBI Reference Sequence: NM_022374.5), for example, but is not limited thereto.

In the present invention, “expression” refers to a process in which a polypeptide is produced from a structural gene. The process may involve transcription of a gene into mRNA and production of a protein through translation of this mRNA into the polypeptide(s). In the present invention, “overexpression” means that the amount of transcript or protein produced is higher than the normal level.

In the present invention, “activity” means that the ATL2 gene or protein performs its function to cause physiological changes.

In the present invention, “inhibition” means a partial (e.g., from 1% to 10% or more, 20 % or more, 30% or more, 40% or more, 50% or more, 60% or more, 70% or more, 80% or more, 90% or more, or 95% or more) or complete decrease in the expression or activity of the ATL2 gene or protein.

In the present invention, the inhibitor of the expression or activity of the ATL2 gene may be one or more selected from the group consisting of microRNA (miRNA), small interference RNA (siRNA), short hairpin RNA (shRNA), a peptide nucleic acid (PNA), an antisense nucleotide, and a ribozyme, which specifically bind to the mRNA of the ATL2 gene and may be the siRNA according to an embodiment or an experimental example of the present invention, but is not limited thereto.

The siRNA may consist of a 15 to 30-mer sense sequence selected from the base sequence of the mRNA of the ATL2 gene and an antisense sequence complementary to the sense sequence, and the sense sequence is not specifically limited. In the present invention, the siRNA may comprise the base sequence represented by SEQ ID NO: 3 or SEQ ID NO: 4, and variants of the base sequence represented by SEQ ID NO: 3 or 4 may also be included within the scope of the present invention. The above-mentioned variants may include functional equivalents of the siRNA represented by SEQ ID NO: 3 or 4 according to the present invention, such as variants in which a part of the sequence represented by SEQ ID NO: 3 or 4 may be modified by deletion, substitution, or insertion to enable the variants to function identically to the siRNA knocking down the ATL2 gene comprising the base sequence represented by SEQ ID NO: 3 or 4. Specifically, the siRNA may include a sequence having a sequence homology of 70% or more, preferably 80% or more, 81%, 82%, 83%, 84%, 85%, 86%, 87%, 88%, 89%, 90%, 91%, 92%, 93%, 94%, 95%, 96 %, 97%, 98%, 99%, or 100% to the base sequence represented by SEQ ID NO: 3 or 4. “The percentage of the sequence homology” for a polynucleotide or amino acid may be determined by comparing two optimally arranged sequences and comparison areas.

In the present invention, the inhibitor of the expression or activity of the ATL2 protein may be one or more selected from the group consisting of compounds, peptides, peptide mimetics, substrate analogs, aptamers, and antibodies, which specifically bind to the ATL2 protein, but is not limited thereto.

The compounds may include all compounds capable of specifically binding to the ATL2 protein and inhibiting its expression or activity.

In the present invention, “aptamer” means a single-stranded nucleic acid, such as DNA, RNA, or modified nucleic acid, having a stable tertiary structure by itself and being able to bind to a target molecule with high affinity and specificity, and it may be possible to develop aptamers for various target substances such as proteins, sugars, dyes, DNA, metal ions, and cells by a method called a systematic evolution of ligands of exponential enrichment (SELEX). The aptamer may specifically bind to a target and modulate the activity of the target, and may block the function of the target by binding thereto, for example.

In the present invention, “antibody” refers to a protein molecule that specifically binds to an antigenic site. The antibodies can be produced by methods commonly practiced in the industry, such as fusion methods, recombinant DNA methods, or phage antibody library methods. In some embodiments, the antibodies or fragments of the antibody may be from different organisms, including humans, mice, rats, hamsters, rabbits, camels, etc. The antibody according to the present invention may be a monoclonal or polyclonal antibody, an immunologically active fragment, an antibody heavy chain, a humanized antibody, an antibody light chain, a genetically engineered single chain Fv molecule, a chimeric antibody, and the like.

In the present invention, the inhibitor of the expression or activity of the ATL2 gene or protein may inhibit the binding between endoplasmic reticulum (ER) and mitochondria in the brain, but is not limited thereto.

In the present invention, the inhibitor of the expression or activity of the ATL2 gene or protein may inhibit mitochondrial superoxide production, but is not limited thereto.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer’s solution, a dextrose solution, a dextrose+sodium chloride solution, PEG, lactated Ringer’s solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO3) carbon dioxide gas, sodium metabisulfite (Na2S2O5), sodium sulfite (Na2SO3), nitrogen gas (N2), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter + cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As another aspect of the present invention, the present invention may provide a food composition for preventing or alleviating Alzheimer’s disease, comprising the inhibitor of the expression or activity of the ATL2 gene or protein as an active ingredient, and the food composition may be a health functional food composition, but is not limited thereto.

The inhibitor of the expression or activity of the ATL2 gene or protein according to the present invention may be used by adding the inhibitor of the expression or activity of the ATL2 gene or protein as is to food or may be used together with other foods or food ingredients, but may be appropriately used according to a typical method. The mixed amount of the active ingredient may be suitably determined depending on the purpose of use thereof (for prevention or alleviation). In general, when a food or beverage is prepared, the inhibitor of the expression or activity of the ATL2 gene or protein of the present invention is added in an amount of 15 wt% or less, preferably 10 wt% or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above-mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

As still another aspect of the present invention, the present invention may provide a composition for diagnosing Alzheimer’s disease, comprising, as an active ingredient, an agent for measuring the level of the expression or activity of the ATL2 gene or protein.

In addition, the present invention may provide a kit for diagnosing Alzheimer’s disease, comprising the composition for diagnosis.

In the present invention, “diagnosis” means confirming the presence or characteristics of a pathological state. For purposes of this invention, the diagnosis is to determine whether a subject has Alzheimer’s disease.

In the present invention, “measurement” means both detecting and confirming the presence (expression) of a target substance and detecting and confirming a change in the level of the presence (expression) of the target substance. The measurement may be performed by both qualitative methods (analysis) and quantitative methods without limitation in methods. Types of the qualitative and quantitative methods for measurement for determining whether the ATL2 gene or protein is present are well known in the industry, and the experimental methods described in the present invention are included therein.

In the present invention, the agent for measuring the level of the expression or activity of the ATL2 gene may be one or more selected from the group consisting of primers, probes, and antisense nucleotides that specifically bind to the mRNA of the ATL2 gene, but is not limited thereto.

In the present invention, a “primer” is a short single-stranded oligonucleotide that serves as a starting point for DNA synthesis. The primer may specifically bind to a polynucleotide as a template with suitable buffer and at an appropriate temperature, and DNA may be synthesized by DNA polymerase adding a nucleoside triphosphate having a base complementary to the template DNA to the primer and linking them together. Primers generally consist of 15 to 30 nucleotide sequences, and the melting temperature (Tm) at which they bind to the template strand varies depending on the composition and length of the bases. The sequence of the primer may not have to be completely complementary to a part of the base sequence of the template. It may be sufficient that the sequence of the primer has a length and complementarity suitable for the purpose of measuring the amount of mRNA by amplifying a specific section of mRNA or complementary DNA (cDNA) by synthesizing DNA. Therefore, in the present invention, it may be possible that a pair of primers is easily designed by referring to the nucleotide sequence of the cDNA or genomic DNA of the mRNA. The primers for the amplification reaction may correspond to a set (pair) of primers that complementarily bind to a template at one end (sense) and a template at the other end (antisense) of a specific section of the mRNA to be amplified, respectively.

In the present invention, “probe” refers to a fragment of a polynucleotide, such as RNA or DNA that can specifically bind to mRNA, cDNA, DNA, etc. of a specific gene and has a length of several to several hundred base pairs, and is labeled so that it is possible to check whether a mRNA or cDNA to be bound is present, the level of the expression thereof, etc. Conditions for the selection and hybridization of the probe may be appropriately selected according to technologies known in the industry. For example, the probe may be used in a diagnostic method to detect alleles. The diagnostic method may include detecting methods based on hybridization of nucleic acids such as the Southern blot, and the probe bound to a substrate of a DNA chip in advance may be used in the method based on a DNA chip.

In the present invention, the primer or probe may be chemically synthesized by a phosphoramidite solid support synthesis method or other well-known methods. In addition, the primer or probe may be modified in various ways by methods known in the industry, as long as hybridization with a polynucleotide to be detected is not hindered. Examples of such modifications may include methylation, capping, substitution of one or more homologs of a natural nucleotide, and modifications between nucleotides, such as uncharged conjugates (e.g., methyl phosphonates, phosphotriesters, phosphoroamidates, carbamates, etc.) or charged conjugates (e.g., phosphorothioate, phosphorodithioate, etc.), and combinations of labeling materials using fluorescence or enzymes.

In the present invention, the primer or probe is not limited to a specific sequence as long as it can measure the expression of the ATL2 gene or mRNA.

In the present invention, the agent for measuring the expression or activity level of the ATL2 protein may be an antibody or an aptamer that specifically binds to the ATL2 protein, but is not limited thereto.

In the present invention, “kit” comprises the agent for measuring the expression or activity level of the ATL2 gene or protein and refers to tools for diagnosing Alzheimer’s disease. In addition to the agent for measuring the level of the expression or activity of the ATL2 gene or protein, the kit according to the present invention may comprise other components, compositions, solutions, devices, etc. that are usually required to detect them. In this case, the agent may be applied one or more times without limitation in the number of times. The order in which each agent is applied is not limited, and each agent may be applied simultaneously or separately. In the present invention, the kit may be a PCR kit, a DNA chip kit, a Western blot kit, or a protein chip kit, but is not limited thereto.

In the present invention, the kit may comprise a container, directions, and the agent for measuring the level of the expression or activity of the ATL2 gene or protein. The container may serve to package the agent and may also serve to store and fix. The material of the container may be, for example, plastic or glass bottle, but is not limited thereto.

According to yet another aspect of the present invention, there may be provided a method of providing information to diagnose Alzheimer’s disease, comprising measuring the level of the expression or activity of the ATL2 gene or protein in a sample collected from the brain of a subject.

The present invention may further comprise diagnosing a subject with Alzheimer’s disease when the expression or activity level of the ATL2 gene or protein is higher than that of a normal control group, but is not limited thereto.

According to yet another aspect of the present invention, there may be provided a method of providing information to predict the risk of developing Alzheimer’s disease, comprising measuring the level of the expression or activity of the ATL2 gene or protein in a sample collected from the brain of a subject.

The present invention may further comprise predicting that the risk of developing Alzheimer’s disease is high when the level of the expression or activity of the ATL2 gene or protein is higher than that of a normal control group, but is not limited thereto.

In the present invention, a “sample” can be used without any limitation as long as it is collected from a subject for whom diagnosis of Alzheimer’s disease or prediction of the risk of developing the disease is performed, and may be, for example, cells or tissues obtained by biopsy, etc., blood, whole blood, serum, plasma, saliva, cerebrospinal fluid, various secretions, urine, feces, and the like. According to one embodiment or experimental example of the present invention, the sample may be a cell or tissue collected from the brain, but is not limited thereto.

In the present invention, the subject may be a mammal such as a human or a non-human primate, a mouse, a rat, a dog, a cat, a horse, and a cow, but is not limited thereto.

In the present invention, the level of the expression of the ATL2 gene may be determined by measuring the level of the mRNA expression of the ATL2 gene.

In the present invention, the level of the mRNA expression of the ATL2 gene may be measured by methods known in the industry, such as polymerase chain reaction (PCR), reverse transcription polymerase chain reaction (RT-PCR), competitive RT-PCR, qRT-PCR, real-time PCR (quantitative PCR, quantitative real-time PCR), RNase protection assay (RPA), the northern blotting, and a DNA chip-based method, but the present invention is not limited thereto.

In the present invention, the level of the expression of the ATL2 protein may be measured by methods known in the industry, such as the western blot, enzyme linked immunosorbent assay (ELISA), radioimmunoassay (RIA), radioimmunodiffusion, ouchterlony immunodiffusion, rocket immunoelectrophoresis, tissue immunostaining, immunoprecipitation assay, complement fixation assay, fluorescence activated cell sorter (FACS), and a protein chip-based method, but the present invention is not limited thereto.

Furthermore, according to the present invention, there may be provided a method of screening candidate substances for treatment of Alzheimer’s disease, which comprises the following steps:
treating presenilin-1 (PS1)-overexpressed cells collected from a subject having the Alzheimer’s disease with a candidate substance;
measuring the level of the expression or activity of the ATL2 gene or protein in the cells; and
selecting a candidate substance as a candidate substance for treatment of Alzheimer’s disease when the level of the expression or activity of the ATL2 gene or protein is lower than the level measured before the cells were treated with the candidate substance.

In the present invention, a “candidate substance” refers to an unknown substance that is screened by observing a change in the expression or activity level of the ATL2 gene or protein in order to confirm its effect on the treatment of Alzheimer’s disease and may be selected from a group consisting of nucleotides, DNA, RNA, amino acids, aptamers, proteins, stem cells, stem cell culture solutions, compounds, microbial culture solutions or extracts, natural products, and natural extracts, but is not limited thereto.

According to yet another aspect of the present invention, there may be provided a method of preventing or treating Alzheimer’s disease, comprising administering a composition comprising the inhibitor of the expression or activity of the ATL2 gene or protein as an active ingredient to a subject in need thereof.

According to yet another aspect of the present invention, there may be provided a use of the composition comprising, as an active ingredient, the inhibitor of the expression or activity of the Atlastin 2 (ATL2) gene or protein for preventing or treating Alzheimer’s disease.

According to yet another aspect of the present invention, there may be provided a use of the composition comprising, as an active ingredient, the inhibitor of the expression or activity of the Atlastin 2 (ATL2) gene or protein for preparing drugs for preventing or treating Alzheimer’s disease.

According to yet another aspect of the present invention, there may be provided a method of diagnosing Alzheimer’s disease or predicting the risk of developing the disease, comprising measuring the level of the expression or activity of the Atlastin 2 (ATL2) gene or protein in a sample collected from the brain of a subject.

According to yet another aspect of the present invention, there may be provided a use of the composition comprising, as an active ingredient, the agent for measuring the level of the expression or activity of the Atlastin 2 (ATL2) gene or protein for diagnosing Alzheimer’s disease.

According to yet another aspect of the present invention, there may be provided a use of the composition comprising, as an active ingredient, the agent for measuring the level of the expression or activity of the Atlastin 2 (ATL2) gene or protein for preparing an agent for diagnosing Alzheimer’s disease.

According to yet another aspect of the present invention, there may be provided a method of treating Alzheimer’s disease, which comprises the following steps:
(a) measuring the level of the expression or activity of the ATL2 gene or protein in a sample collected from the brain of a subject;
(b) diagnosing the subject with Alzheimer’s disease when the expression or activity level of the ATL2 gene or protein is higher than that of a normal control group; and
(c) treating Alzheimer’s disease when the subject is diagnosed with Alzheimer’s disease.

In the present invention, an inhibitor of the expression or activity of the ATL2 gene or protein; or therapeutic agent that usually used for Alzheimer’s disease such as Donepezil (Aricept), Rivastigmine (Exelon), and Galantamine (Razadyne) may be administered for treating Alzheimer’s disease, but is not limited thereto.

According to yet another aspect of the present invention, there may be provided a method of characterizing a responder to therapeutic agent of Alzheimer’s disease for treating Alzheimer’s disease,
wherein the method comprise the following steps:
   (a) measuring the level of the expression or activity of the ATL2 gene or protein in a sample collected from the brain of a subject; and
   (b) determining the subject as a responder to therapeutic agent of Alzheimer’s disease when the expression or activity level of the ATL2 gene or protein is higher than that of a normal control group.

According to yet another aspect of the present invention, there may be provided a method of determining/analyzing whether a subject has high susceptibility to development of the Alzheimer’s disease, which comprises the following steps:
measuring the level of the expression or activity of the ATL2 gene or protein in a sample collected from the brain of a subject suspected as Alzheimer’s disease and normal control; and
determining that the risk of developing Alzheimer’s disease is high when the level of the expression or activity of the ATL2 gene or protein is higher than that of a normal control group.

In the present invention, the “subject” refers to a subject in need of treatment of a disease, and more specifically, refers to a mammal such as a human or a non-human primate, a mouse, a rat, a dog, a cat, a horse, and a cow, but the present invention is not limited thereto.

In the present invention, the “administration” refers to providing a subject with a predetermined composition of the present invention by using an arbitrary appropriate method.

In the present invention, the “prevention” means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

In the present invention, when the expression “including” is used, it means that other components may be further included rather than being excluded unless otherwise described. The expression “the step of doing ...” or “the step of ...” used throughout this specification of the present invention does not have the meaning of “the step for ....”

Hereinafter, the desirable embodiments of the present invention will be presented to assist in understanding the present invention. However, the following embodiments are only provided for easier understanding of the present invention, and the content of the present invention is not limited by the following embodiments.

### EXAMPLES

### Example 1. Animal Models

Food and water were continuously fed to a 7-month-old homozygous PS1M146V knock-in mouse and a wild-type littermate of the same gender (male) and genetic background (C57BL/6) under pathogen-free conditions with a 12-hour light/dark cycle. All the procedures involved were reviewed and approved by the animal care and use committee of the NIA. In addition, 7-month-old and 12-month-old 3xTg-AD mice (APP Swedish, MAPT P301L, PSEN1 M146V) and wild-type littermates of the same genetic background (C57BL/6) were continuously fed food and water under pathogen-free conditions with a 12-hour light/dark cycle. All the experiments and procedures were approved by the animal care committee of the Laboratory Animal Research Center at Sungkyunkwan University.

### Example 2. Human Brain Samples

Samples of the inferior parietal lobule obtained from the brains of three Alzheimer’s disease-affected patients enrolled in the autopsy program of the Alzheimer’s Center at the University of Kentucky and three controls of the same age were used. At autopsy, tissue samples were quickly removed, frozen, and stored at a temperature of -80° C.

### Example 3. Database on Aging, Dementia and TBI by the Allen Brain Institute

This database contains RNAseq-derived transcriptome data from 107 subjects from the adult change in thought (ACT) cohort. A detailed description of tissue collection, tissue processing, and generation of quantitative data can be found in the dataset documentation (http://help.brain-map.org/display/aging/Documentation). RNAseq data of each subject’s frontal white matter (FWM) was used for analysis of the level of the expression of genes.

### Example 4. Incubation of Cells

Human (Homo sapiens’) brain glioma H4 cell line stably expressing wild-type (WT) PS1 or EOFAD-linked PS1 mutants (A431E, E280A, H163R, M146V, or Δexon9) under the control of a tetracycline inhibitor was provided by Brandon Wustman and Anthony Stevens of Amicus Therapeutics, Inc. (1 Cedar Brrok Drive, Cranbury, U.S.A). Cells were incubated in the DMEM medium (Corning, #10-013-CV) supplemented with 10% fetal bovine serum (Gibco, #12483020), 50 µg/ml Zeocin (Invitrogen, #R25001), and 2.5 µg/ml blasticidin (Sigma-Aldrich, #15205). The incubation was continued at a temperature of 37° C. with 95% O₂ and 5% CO₂.

### Example 5. Preparation of Tissue Samples

Preparation of tissue samples was performed as previously reported (Sci Adv. 2021 Jan 13;7(3):eabd3207.). That is, a mouse was anesthetized with Zoletil (Virbac) and Rompun (Bayer) and perfused with 0.9 % NaCl phosphate buffered saline (PBS) (PBS; P3813). The brain of the mouse was dissected to isolate the hippocampus, which was flash frozen in liquid nitrogen and stored at -80° C. until it was analyzed.

### Example 6. Western Blot Analysis

Cells were collected using the T-PER® tissue protein extraction reagent (Thermo Scientific, #78510) along with the protease/phosphatase inhibitor cocktail (Biovision, #K276-1), and were analyzed through the western blot as mentioned in the previous study (J Gerontol A Biol Sci Med Sci. 2021 Jan 1;76(1):23-31.).

Proteins were quantified with the BCA protein assay kit (Thermo Fisher Scientific, #23225) using the xMark microplate spectrophotometer (Bio-Rad, Hercules, CA, USA) at the BIORP, the Korea Basic Science Institute (KBSI). Samples were then separated from SDS-polyacrylamide gels and carried to PVDF membranes. The ratio of the SDS-polyacrylamide gel was adjusted based on the molecular weight of a target protein. An 8% SDS-polyacrylamide gel was used for ATL1, ATL2, ATL3, Tau-13, BACE1, MFN2, OPA1, DRP1, and β-actin, and a 12% SDS-polyacrylamide gel was used for PS1 and APP-CTF. Blots were blocked in 5% non-fat dry milk for one hour at room temperature before being incubated overnight with a primary antibody against ATL1(PA5-85682, Invitrogen), ATL2(PA5-90788, Invitrogen), ATL3(PA5-88408, Invitrogen), PS1(5643, Cell Signaling), APP-CTF(A8717, Sigma), Tau-13(835201, Biolegend), BACE1(5606, Cell Signaling), MFN2(sc-100560, Santa Cruz), OPA1(612606, BD Biosciences), DRP1(sc-32898, Santa Cruz), and β-actin (A5316, Sigma-Aldrich). ATL1, ATL2, ATL3, PS1, APP-CTF, Tau-13, BACE1, and OPA1 antibodies were diluted at a ratio of 1:1000, MFN2 and DRP1 antibodies were diluted at a ratio of 1:200, and β-actin antibodies were diluted at a ratio of 1:10000. Membranes were incubated with an HRP-conjugated secondary antibody for one hour at room temperature, and signals were detected using ECL solution (Pierce, Rockford, IL, USA). Quantification by the western blot bands was performed based on the ImageJ program.

### Example 7. Analysis of Images of ER and Mitochondria

H4^{PS1} cell lines were grown in a DMEM medium supplemented with 10 % fetal bovine serum, 50 µg/ml Zeocin, and 2.5 µg/ml blasticidin. To induce PS1 mutants, each cell line was treated with 100 ng/ml of tetracycline (Sigma-Aldrich, #T7660) for five days. Cells were plated on Nunc Lab-Tek chambered coverglasses 24 hours prior to passaging and imaging. The cells were incubated in Hank’s Balanced Salt Solution (HBSS) containing 100 nM MitoTracker® Red (Thermo Fisher Scientific, #M7512) and 1 µM ER-Tracker® Green (Thermo Fisher Scientific, #E34251) for 20 minutes at 37° C. and with 5% CO₂ according to the manufacturer’s instructions. For fixation, the cells were washed twice with PBS for five minutes each time, fixed with 4% paraformaldehyde (PFA) for 10 minutes, and washed twice with the PBS (five minutes each time). Slides were mounted in the antifade mounting medium with DAPI (VECTASHIELD, #H-1200), and, for the imaging of live cells, HBSS was incubated for 20 minutes and then replaced with a new probe-free medium. Confocal imaging was performed using the LSM700 (Carl Zeiss, Gottingen, Germany). A 405 nm laser was used for the DAPI (Ex/Em = 365/468), a 488 nm laser was used for the ER-Tracker™ Green (Ex/Em = 504/511), and a 555 nm laser was used for the MitoTracker™ Red (Ex/Em = 579/599). Images were then acquired, and a fluorescence intensity profile and a weighted colocalization coefficient were analyzed using the Zeiss ZEN software. The weighted colocalization coefficient was calculated based on the same equation as that of Mander’s colocalization coefficient, but a value of each pixel was identical to an intensity value.

### Example 8. Analysis of Microstructure Through Transmission Electrons

Each H4^{PS1} cell line was fixed, processed, and visualized in the previously mentioned manner (J Physiol. 2000 Dec 15;529 Pt 3(Pt 3):553-64.).

Samples were prepared using ultrathin sections (80 nm) and stained with uranyl acetate and sodium bismuth. The sections were observed with a transmission electron microscope (JEM-1010, JEOL, Tokyo, Japan).

### Example 9. Detection of Mitochondrial Superoxide

Mitochondrial superoxide was detected using a fluorescent MitoSOX™ Red (Invitrogen, #M36008). Cells were incubated with a 2 µM MitoSOX™ Red for 30 minutes at 37° C. with 5% CO₂ before being washed with PBS. Fluorescence was detected using a confocal microscope (Carl Zeiss, LSM700). A 405 nm laser was used for the Hoechst (Ex/Em = 361/497) and a 555 nm laser was used for the MitoSOX™ Red (Ex/Em = 510/580).

### Example 10. Activity of Complex I

The activity of complex I of mitochondria in each H4^{PS1} cell line was observed using the complex I enzyme activity microplate assay kit (Abcam, #ab109721) according to the instructions for kits. That is, 200 µg of protein was incubated in culture buffer for three hours at room temperature and washed three times with 1x buffer. Then, an assay buffer was added, and samples were analyzed on the Synergy HTX Multi-Mode Microplate Reader (BioTek Instruments, Inc, USA) (Abs: 450 nm, 45 sec. interval for 30 minutes, shaking the samples between each reading).

### Example 11. Peroxidase Activity

The peroxidase activity in each H4^{PS1} cell line was observed using the EZ-Hydrogen Peroxide/Peroxidase assay kit (DoGenBio, #DG-PER500) according to the manufacturer’s instructions. That is, 50 µl of a sample and HRP standard solution were incubated with 50 µl of Oxi-Probe/H₂O₂ working solution in a dark room at room temperature for 30 minutes. Each well was analyzed on the Synergy HTX multimode microplate reader (Abs: 560 nm).

### Example 12. Analysis of Hippocampus

The XF24 (Agilent, Santa Clara, USA), an analyzer for the hippocampus, was used to monitor an oxygen consumption rate (OCR) in real time according to the instructions for kits. That is, each H4^{PS1} cell line was injected into an XF24 cell culture plate two days before the experiment. One day before the experiment, a 1 ml XF meter was added to each well of the XF cartridge, and each H4^{PS1} cell line was incubated overnight at 37° C. with 0% of CO₂ in a humidified atmosphere. Then, cells were washed with PBS 30 minutes prior to the experiment, and 625 µl of XF assay medium was added to each well before the cells were incubated at 37° C. with 0% of CO₂ in a humidified atmosphere for 30 minutes. For the assay for XF cell mito stress, the XF assay medium was supplemented with 5 mM glucose and 2 mM glutamine, and, after a 15-minute equilibration time, the OCR was measured four times after addition of compounds, every 8.30 minutes (3 minutes after mixing, waiting for 2 minutes, and measuring for 3.30 minutes). Other compounds were added to the injection port of the XF cartridge at 10x final concentration and diluted in the XF assay medium prior to the experimentation.

### Example 13. Microarray Analysis

A mouse was sacrificed by cervical dislocation, and its hippocampus was removed and flash frozen. Tissues were processed with the Bead Beater (Bio-Spec, Bartlesville, OK, USA), and RNA was purified using the RNEasy mini kit (Qiagen, Valencia, CA, USA). The purified RNA was evaluated for quality and quantity with the Bioanalyzer (Agilent Technologies, Palo Alto, CA, USA). Probe preparation and hybridization were performed as reported in the previous study (Proc Natl Acad Sci USA. 2000 Aug 1;97(16):9127-32.). That is, 5 µg of each RNA sample was used for PCR reaction with the 32^{P}-dCTP (Valeant, Costa Mesa, CA, USA), and radiolabeled cDNA was allowed to hybridize to mouse NIA 17 K cDNA filters overnight at 43° C. The hybridized filters were then washed and placed under an imaging screen for three days before images were developed and scanned. Data were extracted with the ArrayPro software (Media Cybernetics, San Diego, CA, USA).

### Example 14. Selection of Differentially Expressed Genes (DEGs) and Analysis of Function Thereof

All data were processed by the z-score transformation. Genes with an average intensity greater than zero under both conditions were identified first. Then, genes with a z-ratio equal to or greater than 1.5, genes with a z-ratio less than or equal to -1.5, and genes with a P value less than 0.05 were selected as differentially expressed genes (DEGs). The z-ratio is a measure of fold change between comparisons. All gene lists were annotated with the RefSeq-release23 and the Unigene database9. A heat map for values of the expression of genes and the Volcano plots for z-ratios and P-values between selected samples were drawn by an internal R script. GO terms abundant in the DEGs were identified using the g:Profiler (version 0.6.7).

### Example 15. Transient Transfection

Cells were injected into 6-well dishes at 70% confluence using the OptiMEM and the INTERFERin (Polyplus-transfected) according to the manufacturer’s instructions, and were transfected with the siRNA (Bioneer, Daejeon, Korea) against the ATL2. The transfected cells were maintained for 24 hours before further experiments, and the sequences of the siRNA are shown in Table 1 below.

TABLE 1

| | Sequence | SEQ. ID NO. |
|---|---|---|
| ATL2 sense | 5′-UAG AGU UUG UUA CAG ACU G-3′ | 3 |
| ATL2 antisense | 5′-CAG UCU GUA ACA AAC UCU A-3′ | 4 |

### Example 16. Quantitative Real-Time PCR (qPCR)

The total RNA was extracted from the H4^{PS1} cell line with the RNAiso plus (TaKaRa, Shiga, Japan) and reverse transcribed into cDNA using the PrimeScript RT Master Mix (TaKaRa). In addition, the qPCR was performed using the TB Green PCR Kit (TaKaRa) to detect mRNA expression of ATL1, ATL2, and ATL3, and GAPDH was used as a reference gene. The used qPCR primer sequences are shown in Table 2 below.

TABLE 2

| | Sequence | SEQ. ID NO. |
|---|---|---|
| ATL1 forward | 5′-CCC TGT GCA CTT GGG CAT AT-3′ | 5 |
| ATL1 reverse | 5′-TTG TAC AAA GCC TGG TCC CAC-3′ | 6 |
| ATL2 forward | 5′-TTG CCA CAT CCT GGT CTT AAA-3′ | 7 |
| ATL2 reverse | 5′-GCA GCA ATG GAA CCA GAT TT-3′ | 8 |
| ATL3 forward | 5′-ACA AGC CCT GAC TTT GAT GG-3′ | 9 |
| ATL3 reverse | 5′-TGC AGC TGC TAA GTT GTT GG-3′ | 10 |
| GAPDH forward | 5′-AGC CAC ATC GCT CAG ACA C-3′ | 11 |
| GAPDH reverse | 5′-GCC CAA TAC GAC CAA ATC C-3′ | 12 |

### Example 17. Measurement of Intracellular ROS

The level of intracellular ROS was measured using the fluorescent dye, chloromethyl 2′,7′-dichloro-fluorescein diacetate (CM-H₂DCFDA) (Invitrogen, #C6827), and it was converted to 2′,7′-dichlorofluorescein (DCF), which becomes highly fluorescent in the presence of an oxidizing agent. That is, 24 hours after each H4^{PS1} cell line was plated on a 96-well plate, cells were cultured for 30 minutes with CM-H₂DCFDA (5 µM) in a serum-free medium. Fluorescence intensity was measured using the Victor X3 plate reader (Perkin Elmer, USA) according to the manufacturer’s protocol (Ex/Em = 492/527).

### Example 18. Measurement of Mitochondrial Membrane Potential and Level of ATP

A mitochondrial membrane potential was measured in the 5,5′,6,6′-tetrachloro-1,1′,3,3′-tetraethyl-benzimidazolylcarbocyanine iodide (JC-1) (BD Biosciences, #551302) or an H4^{PS1} cell line in a 96-well plate with TMRM (tetramethylrhodamine, methyl ester, and perchlorate). In addition, the fluorescence intensities for aggregate and monomeric forms of the JC-1 were measured with the TECAN Infinite 200 multifunctional microplate reader (TECAN, Switzerland) according to the manufacturer’s protocol (JC-1 aggregate: Ex/Em = 525/590; JC-1 monomer: Ex/Em = 490/530), and the fluorescence intensity for the TMRM was measured with the Synergy NEO multimode microplate reader (BioTek Instruments, USA) according to the manufacturer’s protocol (Ex/Em = 548/574). Total cellular ATP levels were detected using the ATP Bioluminescence Detection Kit (Promega, #TB267) according to the manufacturer’s protocol.

### Example 19. Statistical Analysis

The statistical analysis was entirely performed using the GraphPad Prism 8.0 software (GraphPad, La Jolla, CA, USA). All data are presented as mean ± standard error of the mean (SEM). Depending on the variable, differences between groups were compared using either one-way or two-way analysis of variance (ANOVA) with Bonferroni post hoc tests, and the student’s t-test (two-tailed) was used to compare H4^{PS1} cell lines observed before and after PS1 was induced. A P-value < 0.05 was considered statistically significant.

### EXPERIMENTAL EXAMPLES

### Experimental Example 1. Accumulation of APP-CTF in H4^{PS1} Cell Lines as a Result of Inducing PS1 Mutants

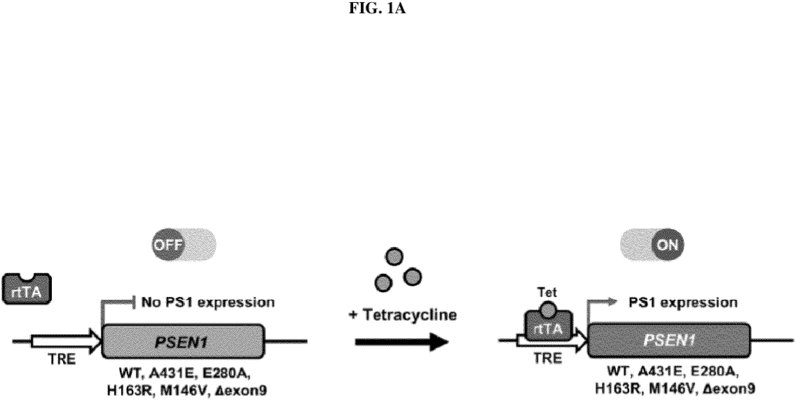

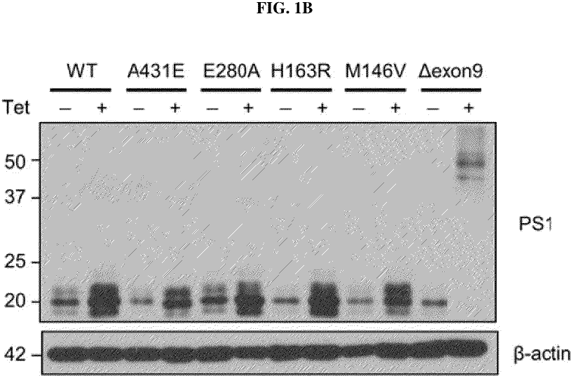

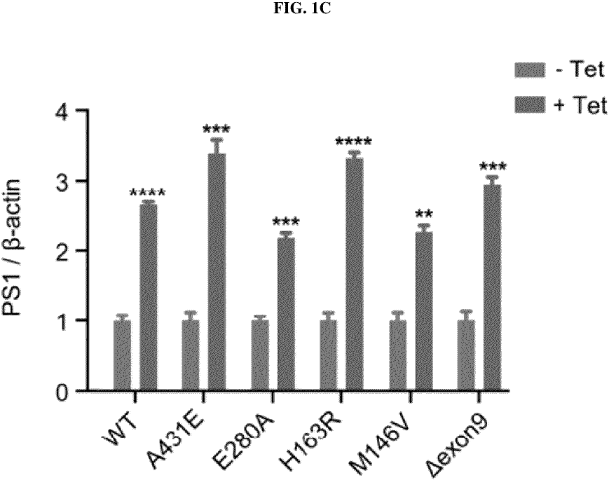

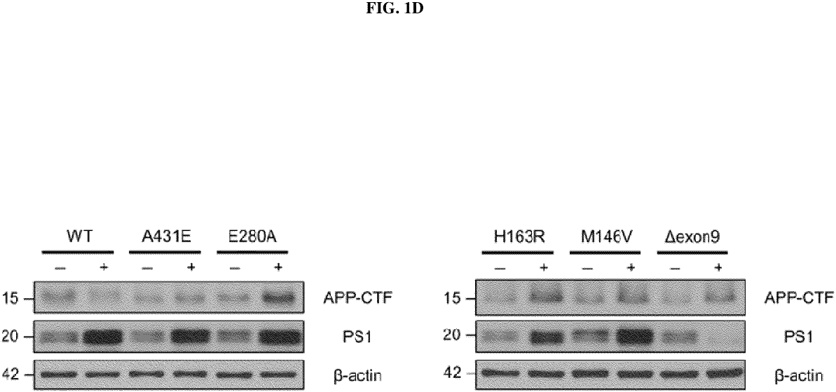

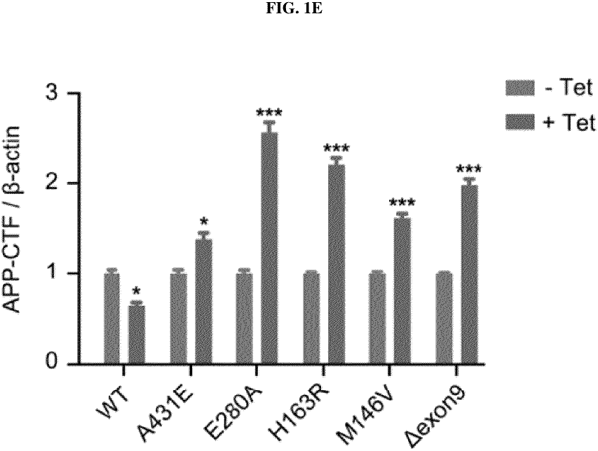

Since PS1 is the catalytic subunit of γ-secretase that cleaves type 1 transmembrane protein, the effect of each PS1 mutant on the γ-secretase activity was examined by first detecting APP C-terminal fragments (APP-CTFs). FIG. 1A schematically shows a system for inducing the PS1 mutants with tetracycline, which is used for such an examination.

FIG. 1B shows the results of performing the western blot on PS1 mutants for PS1 in H4^{PS1} cell lines before and after treatment with tetracycline, and FIG. 1C shows the results of quantifying the expression of the PS1 mutants in FIG. 1B.

As shown in FIGS. 1B and 1C, each PS1 mutant was successfully induced by treatment with 100 ng/ml of tetracycline, and the level of overexpression was observed to be 2.2 to 3.4fold. In the H4^{PS1Δexon9} cell line, 44 kDa full-length PS1 may accumulate upon treatment with tetracycline because deletion of exon 9, which contains an in vivo proteolytic site, may prevent cleavage of the full-length PS1. In addition, as shown in FIG. 1B, it was observed that endogenous PS1 decreased in the H4^{PS1Δexon9} cell line upon treatment with tetracycline. This is consistent with the results in the previous studies reporting that overexpression of exogenous PS1 replaces the endogenous PS1. Through this replacement, it was possible to exclude the potential effects of the endogenous PS1 in the induced H4^{PS1} cell lines.

FIG. 1D shows the results of the western blot on PS1 mutants for APP-CTF in H4^{PS1} cell lines before and after treatment with tetracycline, and FIG. 1E shows the results of quantifying the APP-CTF in FIG. 1D.

Upon treatment with tetracycline, as shown in FIGS. 1D and 1E, the APP-CTF was observed to accumulate in all the five H4^{PS1} mutant cell lines, indicating the γ-secretase activity was attenuated. These results are consistent with the results in the previous studies reporting that the γ-secretase activity is attenuated upon expression of FAD-linked PS1 mutant proteins.

In FIGS. 1C and 1E, “n”=3, “*”P<0.05, “**”P<0.01, and “***”P<0.001.

### Experimental Example 2. Accumulation of Fragmented Mitochondria in H4^{PS1Δexon9} Cell Line as a Result of Inducing PS1 Mutants

Mitochondrion is one of the most dynamic organelles, and can change its size, shape, and location. The mitochondria undergo fusion and fission to maintain their function under metabolic or environmental stress. The fusion brings together the various contents of the mitochondria to relieve stress, while the fission removes damaged mitochondria and creates new ones. Increased mitochondrial fission is seen in neurons from patients with several neurodegenerative disorders, including Alzheimer’s disease (AD).

Therefore, the size and shape of mitochondria in each H4^{PS1} cell line were observed to determine whether PS1 mutants affect the mitochondrial fusion or fission.

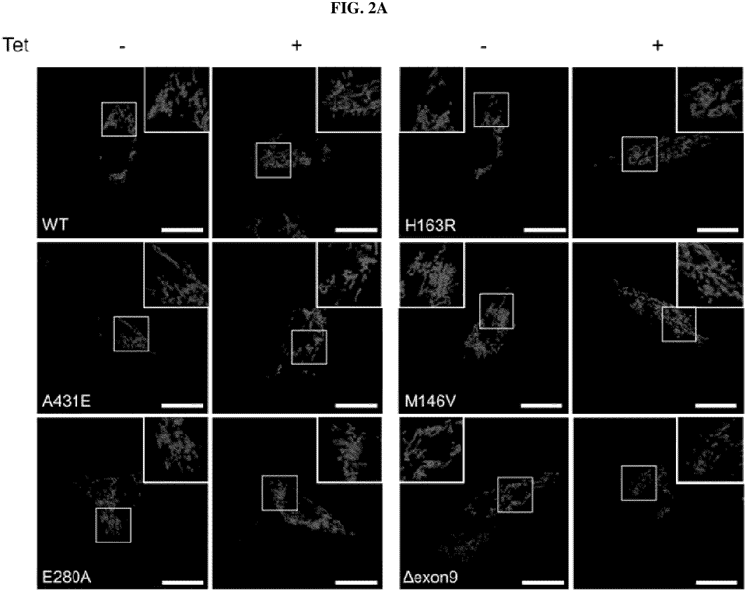

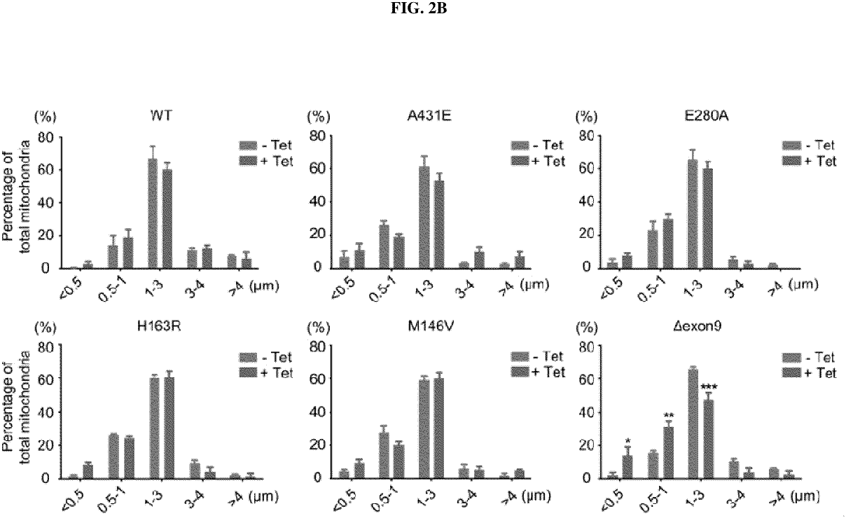

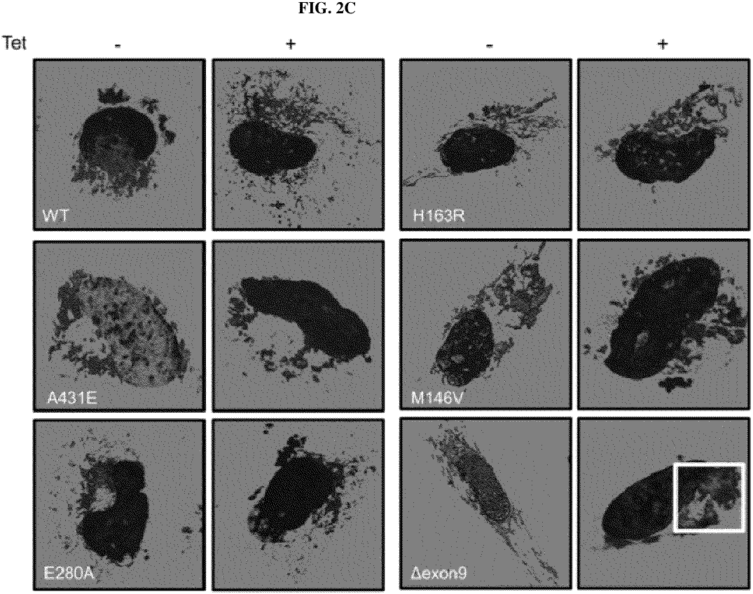

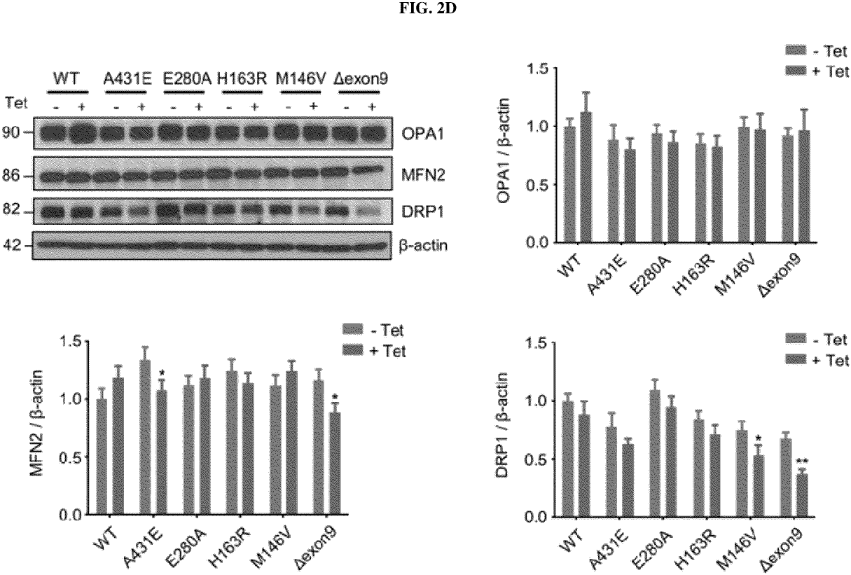

FIG. 2A shows the images of MitoTracker-labeled H4^{PS1} cell lines viewed with a confocal microscope before and after they were treated with tetracycline (scale bar = 20 µm). As shown in FIG. 2A, before overexpressed PS1 mutants were induced, mitochondria with fibers similar in shape to sausages appeared in all the six H4^{PS1} cell lines. However, fragmented mitochondria were increased in the H4^{PS1Δexon9} cell line after the overexpressed PS1 mutants were induced, and the mitochondria were shortened, punctate, and spherical.

FIG. 2B shows the results of quantifying the length of mitochondria in the H4^{PS1} cell lines before and after treatment with tetracycline. 8 to 12 cells were analyzed for each image, and the number of mitochondria per cell was 48 to 121 (n=3, *P<0.05, **P<0.01, ***P<0.001). As shown in FIG. 2B, as a result of measuring the length of mitochondria using a confocal microscope, it was observed that fragmented mitochondria significantly increased in the H4^{PS1Δexon9} cell line after the overexpressed PS1 mutants were induced, and no significant difference in the size of mitochondria was observed in other H4^{PS1} cell lines before and after the overexpressed PS1 mutants were induced.

FIG. 2C shows the distribution of mitochondria in the H4^{PS1} cell lines observed by the z-stack imaging of a confocal microscope (z-axis: 5 µm, 100 slices), and, as shown in FIG. 2C, when the distribution of mitochondria in the H4^{PS1Δexon9} cell line was observed by the z-stack imaging, relatively aggregated mitochondria were observed in the cytoplasm as the overexpressed PS1 mutants were induced.

FIG. 2D shows the results of the western blot on PS1 mutants for mitochondrial dynamics-related proteins (OPA1, MFN2, and DRP1) in the H4^{PS1} cell lines and the results of the quantification thereof. The OPA1 and MFN2 may mediate the mitochondrial fusion, whereas the DRP1 may mediate the mitochondrial fission. It is reported that higher levels of the activity of the DRP1 and mitochondrial fragmentation are observed in the brains of patients with sporadic Alzheimer’s disease (SAD). Previous studies have reported that the dysfunction of Aβ-mediated mitochondria can be ameliorated by the inhibition of the DRP1. As a result of observing the expression levels of proteins related to mitochondrial dynamics, such as Protein1 (DRP1) related to optic atrophy1 (OPA1), mitofusin2 (MFN2), and GTPase dynamin, it was found that, as shown in FIG. 2D, the expression levels of the DRP1 and the MFN2 were significantly decreased in the H4^{PS1Δexon9} cell line after overexpressed PS1 mutants were induced, whereas there was no change in the expression level of the OPA1.

### Experimental Example 3. Increase in the Percentage of Mitochondria Contacting Endoplasmic Reticulum in H4^{PS1} Cell Lines as a Result of Inducing PS1 Mutants

PS1 and PS2 are found in numerous intracellular compartments such as endoplasmic reticulum (ER), Golgi apparatus, plasma membrane, nuclear envelope, endosomes, lysosomes, and mitochondria. In particular, they contain an abundance of MAM, a sub-compartment of the endoplasmic reticulum that is physically connected to the mitochondria. A direct interaction between the endoplasmic reticulum and the mitochondria is possible through the MAM. This interaction plays an essential role in determining the fate of cells by controlling the functions of all other intracellular compartments, so the effect of each PS1 mutant on the interaction between the endoplasmic reticulum and the mitochondria was observed using a confocal microscope.

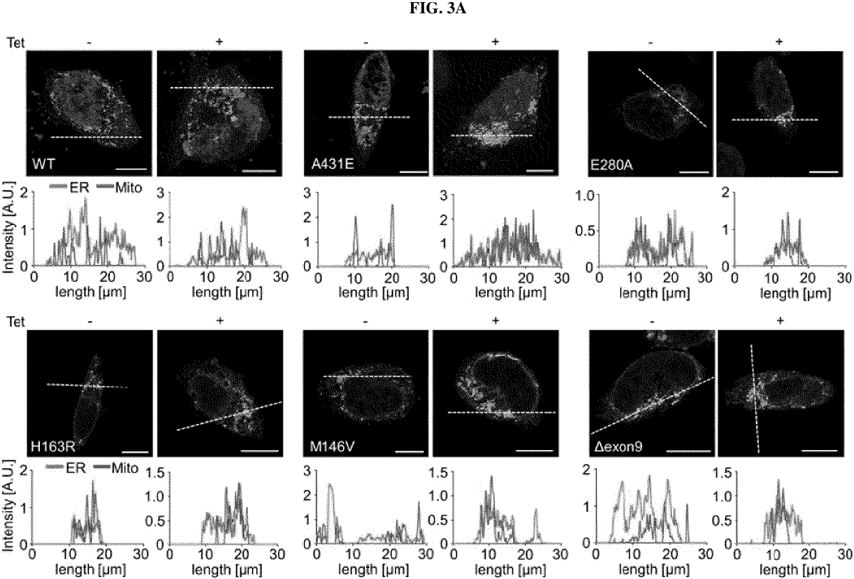

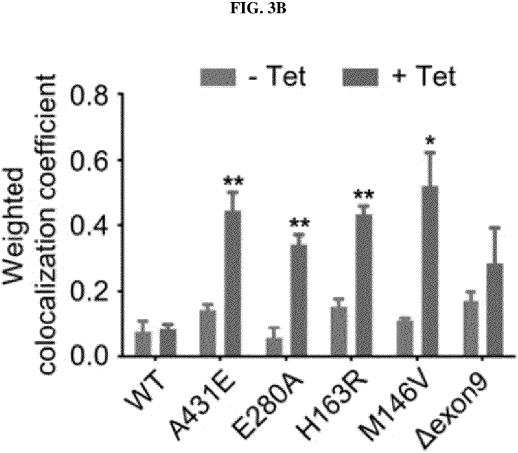

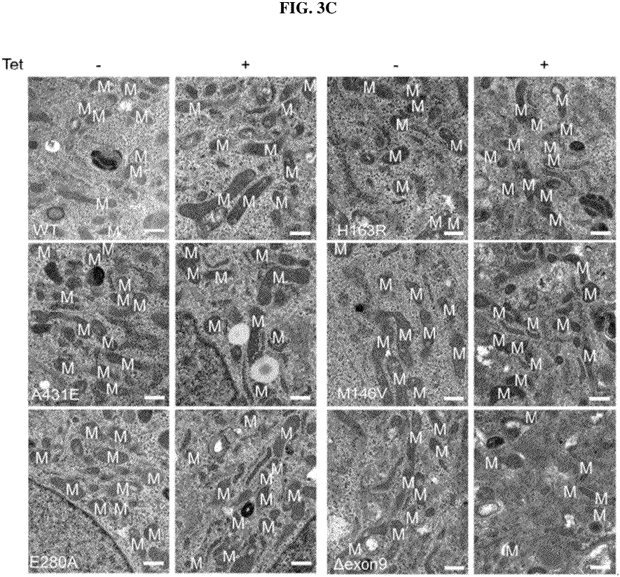

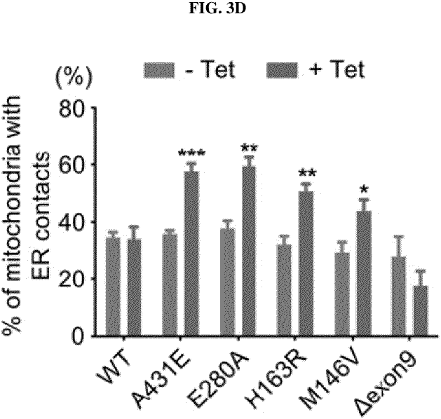

FIG. 3A shows the results of analyzing H4^{PS1} cells labeled with MitoTracker (red) and ERTracker (green) through the confocal imaging and line scan before and after treatment with tetracycline, and FIG. 3B shows the results of observing the weighted colocalization coefficients between mitochondria and endoplasmic reticulum in H4^{PS1} cells before and after treatment with tetracycline. 10 to 14 cells were analyzed for each imaging (n=3, *P<0.05, **P<0.01, ***P<0.001).

As shown in FIG. 3A, after overexpressed PS1 mutants were induced, increased ER-mitochondria colocalization was observed in the H4^{PS1A431E,} H4^{PS1E280A}, H4^{PS1H163R}, and H4^{PS1M146V} cell lines, but this change was not observed in the H4^{PS1WT} and H4^{PS1Δexon9} cell lines. In addition, as shown in FIGS. 3A and 3B, it was found that the fluorescence intensity profile and colocalization significantly increased in the H4^{PS1A431E}, H4^{PS1E280A}, H4^{PS1M146V}, and H4^{PS1H163R} cell lines after the overexpressed PS1 mutants were induced.

In addition, to further confirm the above-mentioned results, analysis by a transmission electron microscope (TEM) was performed on each H4^{PS1} cell line.

FIG. 3C shows the images of the H4^{PS1} cell lines viewed with a transmission electron microscope before and after treatment with tetracycline, and FIG. 3D shows the results of quantifying mitochondria in contact with endoplasmic reticulum in the H4^{PS1} cell lines before and after treatment with tetracycline. 5 to 10 cells were analyzed for each imaging, and the number of mitochondria per cell was 10 to 35.

As shown in FIGS. 3C and 3D, consistent with the colocalization data above, the percentage of the mitochondria in contact with the endoplasmic reticulum was significantly increased in the H4^{PS1A431E,} H4^{PS1E280A}, H4^{PS1H163R}, and H4^{PS1M146V} cell lines after overexpressed PS1 mutants were induced.

### Experimental Example 4. Differential Changes in Mitochondrial Function in H4^{PS1} Cell Lines as a Result of Inducing PS1 Mutants

Most of ROS are produced during mitochondrial respiration, and the production of the ROS causes oxidative damage in numerous diseases including Alzheimer’s disease. The oxidative damage has been observed in the early stages of Alzheimer’s disease even before the onset of plaque and tau pathology. Accordingly, first of all, the level of the production of the ROS in each H4^{PS1} cell line was measured using CM-H₂DCFDA before and after overexpressed PS1 mutants were induced.

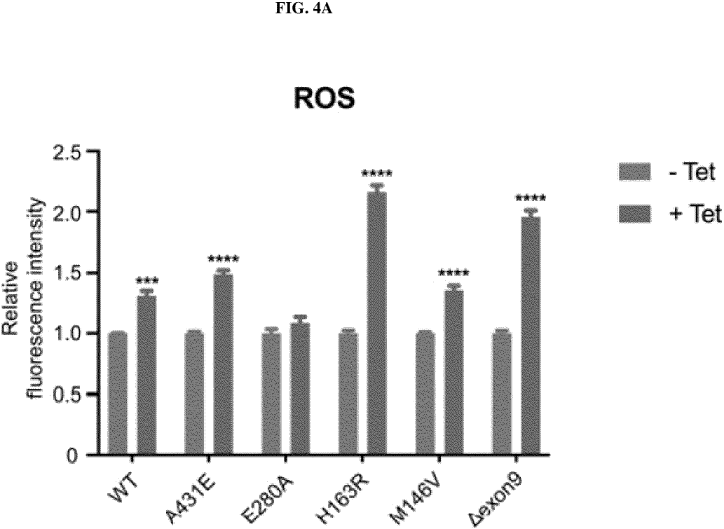

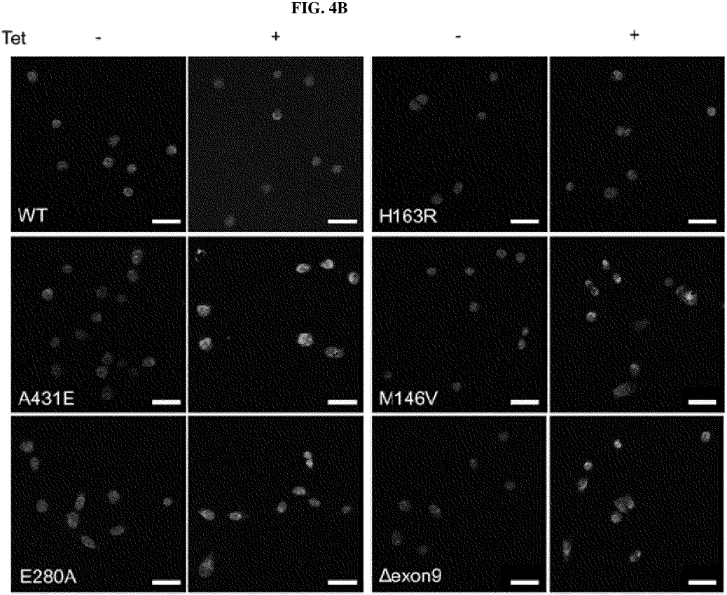

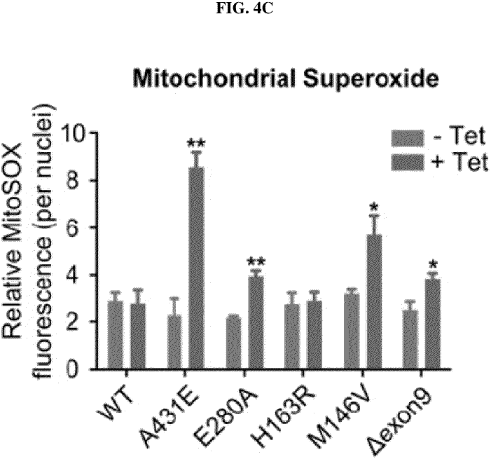

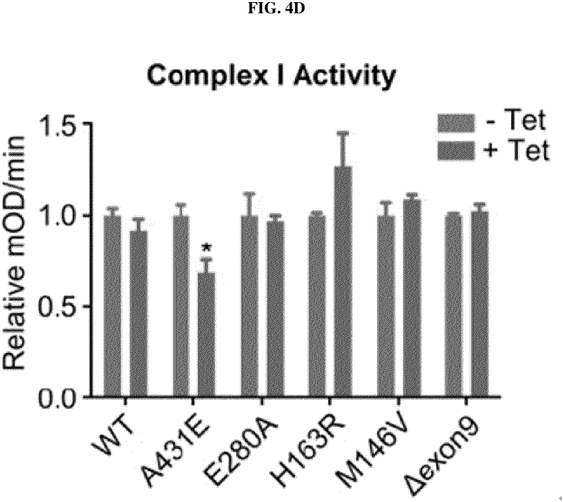

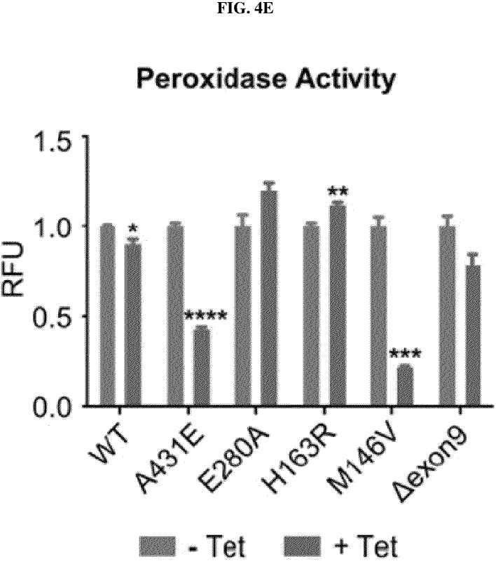

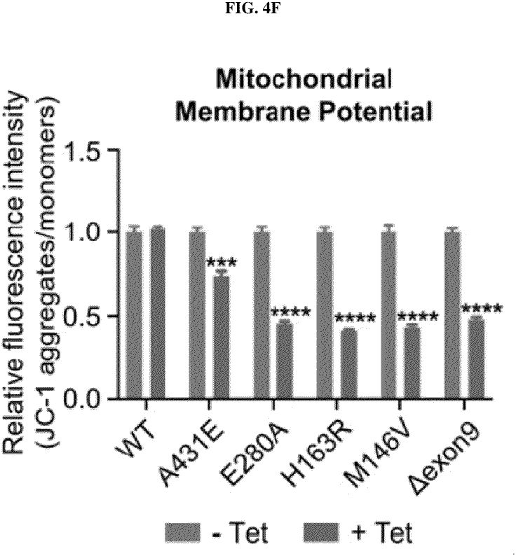

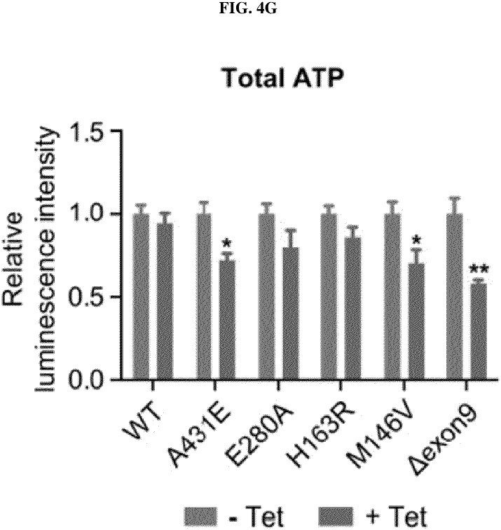

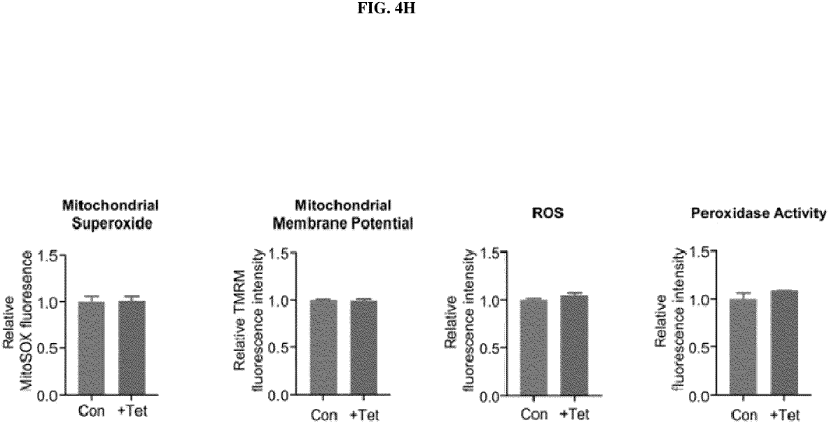

FIG. 4A shows the results of quantifying the production level of the ROS in H4^{PS1} cell lines before and after treatment with tetracycline (n = 4, ***P<0.001, ****P<0.0001), and, as shown in FIG. 4A, it was observed that the production of the ROS increased in all the H4^{PS1} cell lines except for the H4^{PS1E280A} cell line after overexpressed PS1 mutants were induced.

In addition, since superoxide (O₂˙⁻) is a mitochondrial proximal ROS, the production level of the O₂˙⁻ was measured in each H4^{PS1} cell line.

FIG. 4B shows the confocal images of H4^{PS1} cell lines stained with MitoSOX (red) and Hoechst (blue), which were taken before and after treatment with tetracycline, and FIG. 4C shows the results of quantifying the production of mitochondrial superoxide in the H4^{PS1} cell lines before and after treatment with tetracycline. 25 to 51 cells were analyzed for each imaging (n=3, *P<0.05, **P<0.01). As shown in FIGS. 4B and 4C, it was found that mitochondrial O₂˙⁻ was significantly increased in the H4^{PS1A431E}, H4^{PS1E280A}, H4^{PS1M146V}, and H4^{PS1Δexon9} cell lines based on the MitoSOX fluorescence after the overexpressed PS1 mutants were induced.

There are two sources of mitochondrial O₂˙⁻, and they are complex I (NADH coenzyme Q oxidoreductase) and complex III (ubiquinol cytochrome c oxidoreductase). The complex III produces most of the O₂˙⁻ in the heart and lungs, whereas the complex I is the main source of the O₂˙⁻ for the brain. When complex I activity is impaired, NADH accumulates, the potential for reduction of NAD⁺ decreases, and the production of superoxide increases. Such an inverse relationship between the production of the superoxide and the complex I activity has been previously reported. Therefore, it was examined whether the observed increase in the level of the mitochondrial O₂˙⁻ was caused by the abnormal activity of the complex I.

FIG. 4D shows the results of quantifying the activity of complex I of mitochondria in the H4^{PS1} cell lines before and after treatment with tetracycline based on an assay for the activity of complex I enzyme (n=3, *P <0.05), and, as shown in FIG. 4D, the activity of the complex I was significantly reduced in the H4^{PS1A431E} cell line after overexpressed PS1 mutants were induced.

SOD (Superoxide dismutase) catalytically converts O₂˙⁻ produced during respiration into hydrogen peroxide (H₂O₂) and molecular oxygen (O₂). Accumulation of H₂O₂ may have detrimental effects on cells because it may be converted into highly reactive hydroxyl radicals (·OH) through the Fenton reaction in the presence of Fe²⁺. Catalase, an antioxidant enzyme, prevents this by converting H₂O₂ into H₂O and O₂. Unfortunately, the catalase does not exist in mitochondria. Glutathione peroxidase (GPx), an enzyme with peroxidase activity in mitochondria, reduces H₂O₂ to H₂O and lipid hydroperoxides to their corresponding alcohols to protect organisms from oxidative damage. Accordingly, it was examined whether the peroxidase activity was perturbed by the expression of PS1 mutants in each H4^{PS1} cell line.

FIG. 4E shows the results of quantifying the peroxidase activity in the H4^{PS1} cell lines before and after treatment with tetracycline based on the assay for peroxidase (n=3, *P<0.05, **P<0.01, ***P<0.001, ****P<0.0001), and, as shown in FIG. 4E, the peroxidase activity was significantly impaired in the H4^{PS1A431E} and H4^{PS1M146V} cell lines after overexpressed PS1 mutants were induced. In contrast, the peroxidase activity was significantly increased in the H4^{PS1H163R} cell line.

Since the complex I and the peroxidase activity are essential for maintaining the level of mitochondrial membrane potential and ATP, the levels of the mitochondrial membrane potential and the ATP in the H4^{PS1} cell lines were examined before and after PS1 mutants were induced.

FIG. 4F shows the results of quantifying the mitochondrial membrane potential in the H4^{PS1} cell lines before and after treatment with tetracycline based on the Mito-Probe JC-1 assay, and FIG. 4G shows the results of quantifying the level of total ATP in the H4^{PS1} cell lines before and after treatment with tetracycline based on the ATP bioluminescence detection assay (n=4, *P<0.05, **P<0.01, ***P<0.001, ****P<0.0001). As shown in FIG. 4F, the mitochondrial membrane potential was significantly decreased in all the five H4^{PS1} mutant cell lines, and, as shown in FIG. 4G, the level of the ATP was significantly decreased in the H4^{PS1A431E,} H4^{PS1M146V}, and H4^{PS1Δexon9} cell lines.

Previous studies reported that mitochondrial function could also be disrupted just with treatment with tetracycline. To confirm that the impairment of the mitochondrial function observed in each H4^{PS1} cell line was caused by inducing PS1 mutants, not by treatment with tetracycline, the function of some mitochondria in naive H4 cells was evaluated before and after treatment with tetracycline.

FIG. 4H shows the results of observing mitochondrial function in H4^{PS1} cell lines before and after treatment with tetracycline. In order from the left to the right, the first figure shows the results of quantifying the production of mitochondrial superoxide through the MitoSOX (n=3), the second figure shows the results of quantifying mitochondrial membrane potential through the TMRM (n=4), the third figure shows the results of quantifying the production of the ROS through the CM-H₂DCFDA (n=4), and the last figure shows the results of quantifying the peroxidase activity based on the assay for the peroxidase (n=4). As shown in FIG. 4H, it was found that treatment with 100 ng/ml of tetracycline did not affect the production of the mitochondrial superoxide, the mitochondrial membrane potential, the production of the intracellular ROS, and the peroxidase activity. In addition, the concentration of tetracycline used in the research by Moullan et al. was 5 to 10 times higher than the concentration of tetracycline used in this research (Moullan N et al., Cell Rep. 2015;10:1681-91.).

### Experimental Example 5. Changes in Mitochondrial Bioenergetics as a Result of Inducing PS1 Mutants

To determine whether a PS1 mutant affect mitochondrial respiration, the oxygen consumption rate (OCR) was measured while sequentially treating compounds that modulate mitochondrial activity using a flux analyzer in hippocampal XF24 cells, and the functional bioenergetic ability of mitochondria in each H4^{PS1} cell line was examined.

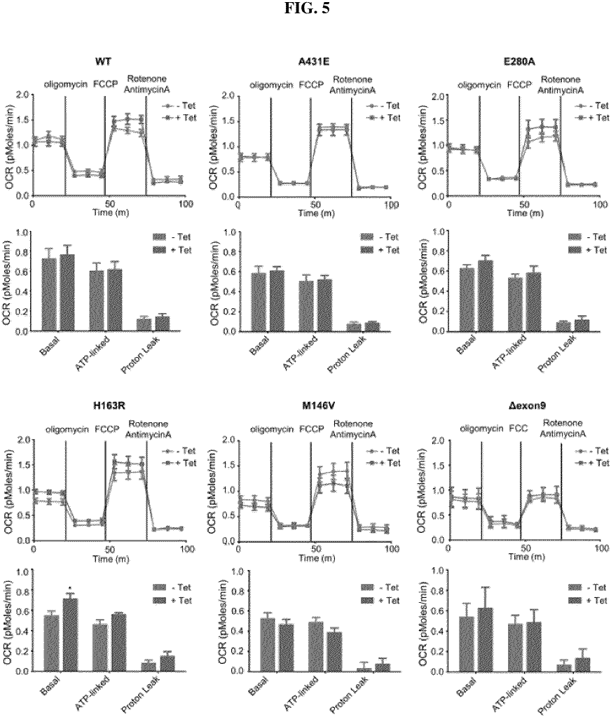

FIG. 5 shows the results of analyzing hippocampal oxygen consumption by observing basal respiration, ATP-linked respiration, and proton leak in H4^{PS1} cell lines before and after treatment with tetracycline (n=4, *P<0.05), and, as shown in FIG. 5, in most of the H4^{PS1} cell lines except for the H4^{PS1H163R} cell line, there was no significant difference in basal OCRs measured before and after overexpressed PS1 mutants were induced. The basal OCR was significantly increased in the H4^{PS1H163R} cell line after the overexpressed PS1 mutants were induced.

The basal respiration consists of two components: oxygen consumption for ATP synthesis and oxygen consumption due to spontaneous proton leak through mitochondrial inner membranes. By the addition of oligomycin, an ATP synthase inhibitor, these two components are separated. There were no significant differences in the ATP-linked respirations and the proton leaks in all the H4^{PS1} cell lines, measured before and after the overexpressed PS1 mutants were induced.

### Experimental Example 6. Examination of Gene Expression Profile of PS1M146V Knock-In Mice

To investigate the effect of the overexpression of PS1 mutants on gene expression profiles, the microarray analysis was performed on hippocampal samples from wild-type and PS1M146V knock-in mice. A total of 16,896 raw reads were obtained using the mouse NIA 17k cDNA filter, and the raw reads were preprocessed and analyzed with the DIANE 1.0 to identify differentially expressed genes (DEGs). As a result, a total of 409 DEGs were finally identified, and 203 (49.63 %) of the DEGs were upregulated and 206 (50.37 %) were downregulated.

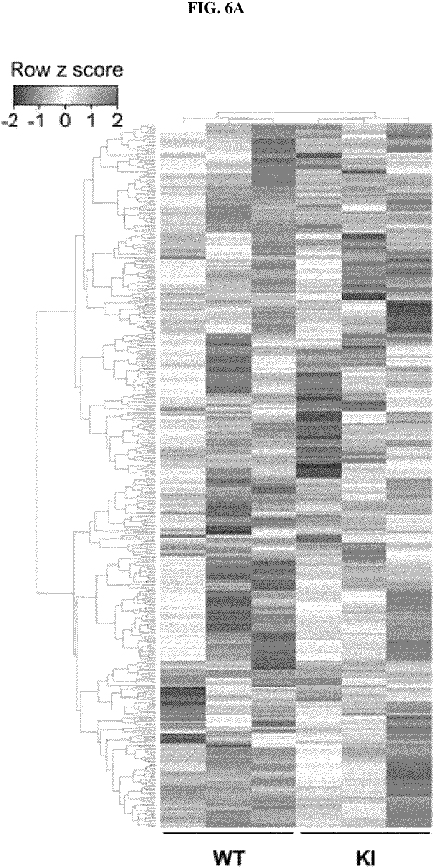

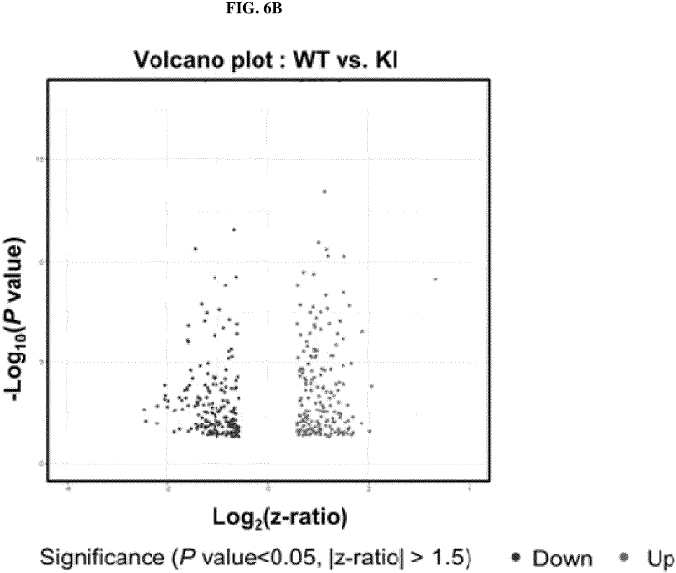

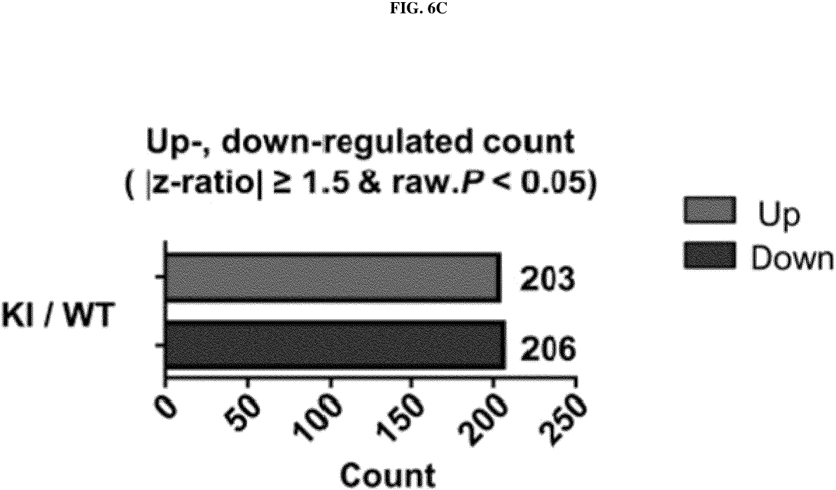

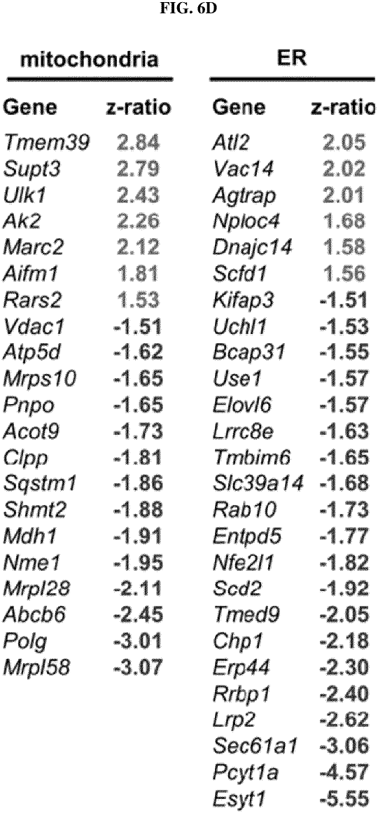

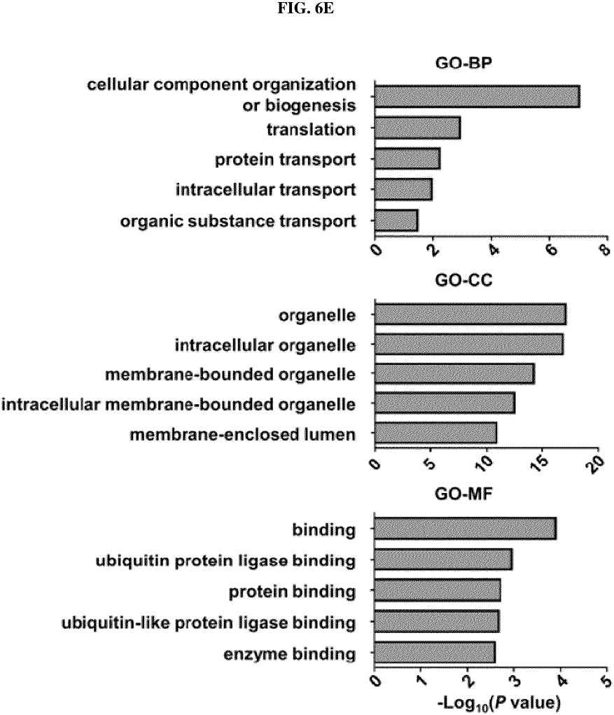

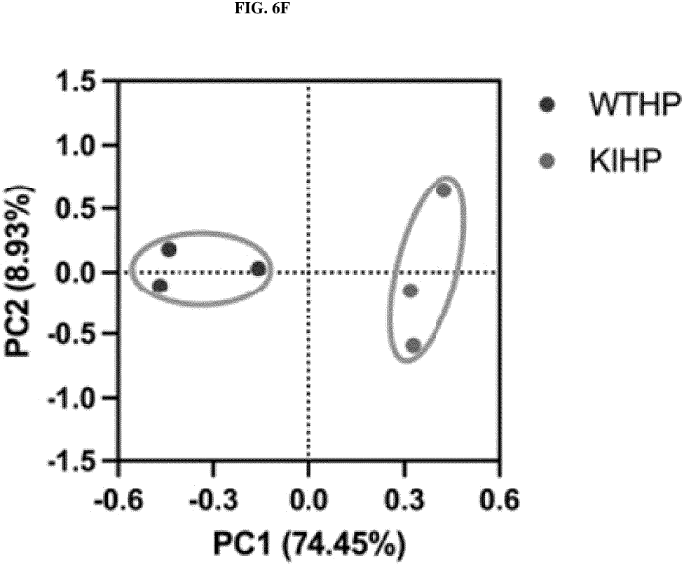

FIGS. 6A to 6E relate to the expression profile of hippocampal genes in the PS1M146V knock-in mice. FIG. 6A shows hierarchical clustering, FIG. 6B shows the Volcano plot of DEGs, FIG. 6C shows the number of counts differentially upregulated and downregulated based on a z-ratio and P-value, FIG. 6D shows a list of DEGs related to mitochondria or endoplasmic reticulum, and FIG. 6E shows an analysis of the gene ontology and the pathway frequency of the DEGs (BP: biological process; CC: cellular component; and MF: molecular function).

As shown in FIGS. 6A to 6C, the hierarchical clustering showed that the gene expression profile of the PS1M146V knock-in mice was distinct from that of the wild-type mice. Then, as shown in FIGS. 6D, 21 DEGs related to mitochondria and 26 DEGs related to ER were identified, and, for further understanding of the functions of the DEGs, the analysis of the gene ontology (GO) was performed using the g:Profiler (ver. 0.6.7). As a result, as shown in FIG. 6E, the DEGs were significantly abundant in biological processes such as cell component organization or biogenesis, translation, protein transport, intracellular transport, and organic substance transport. The DEGs were also significantly abundant in cellular components such as organelles, intracellular organelles, membrane-bound organelles, intracellular membrane-bound organelles, and membrane-enclosed lumens. Finally, the DEGs were significantly abundant in molecular functions such as binding, ubiquitin protein ligase binding, protein binding, ubiquitin-like protein ligase binding, and enzyme binding.

In addition, a principal component analysis (PCA) was performed to confirm that the differential expression data were not driven by one particular one out of the three animals used for the genotypes.

FIG. 6F shows the results of the analysis of the main components of the hippocampus of the wild-type and PS1M146V knock-in mice, and, as shown in FIG. 6F, it was observed that there was a clear difference between the results for the group of wild-type mice and those for the group of PS1M146V knock-in mice.

In addition, since presenilin1 plays an important role in regulating neural differentiation by influencing the notch signaling pathway, it was examined whether there were changes in cell type ratios in the brains of the PS1M146V knock-in mice, and expression levels of genes encoding specific markers for neurons, microglia, and astrocytes were analyzed. As a result, as shown in Table 3 below, all the markers were observed to have a P-value greater than 0.10 and a z-ratio greater than -1.3 and less than 1.3, indicating that the proportions of neurons, microglia and astrocytes in the brains of the PS1M146V knock-in mice did not change.

TABLE 3

Neuron

| Gene | z-ratio | P value |
|---|---|---|
| Cath1 | 0.11 | 0.33 |
| Tubb3 | -0.55 | 0.33 |
| Mapt | 0.17 | 0.90 |
| Nefl | 0.18 | 0.59 |

Microglia

| Gene | z-ratio | P value |
|---|---|---|
| HexB | -1.29 | 0.12 |
| Lgas3 | -0.40 | 0.64 |
| Csf1r | 0.13 | 0.64 |
| Astrocyte | | |
| Gene | z-ratio | P value |
| Slc1a3 | 0.59 | 0.26 |
| Gja1 | 0.19 | 0.71 |
| Aldo3 | 0.35 | 0.62 |

### Experimental Example 7. ATL2 Affecting Endoplasmic Reticulum-Mitochondria Contact In H4^{PS1M146V} Cell Line Abnormally Elevated After Mutagenesis

The examination also focused on ATL2, the most upregulated ER-related gene of the PS1M146V knock-in mice, among the 26 DEGs related to endoplasmic reticulum. The ATL2 gene encodes the endoplasmic reticulum-resident membrane bound GTPase Atlastin 2 (ATL2), which mediates endoplasmic reticulum membrane fusion and tethering. Therefore, first of all, it was examined whether ATL2 expression increased in each H4^{PS1} cell line by inducing PS1 mutants.

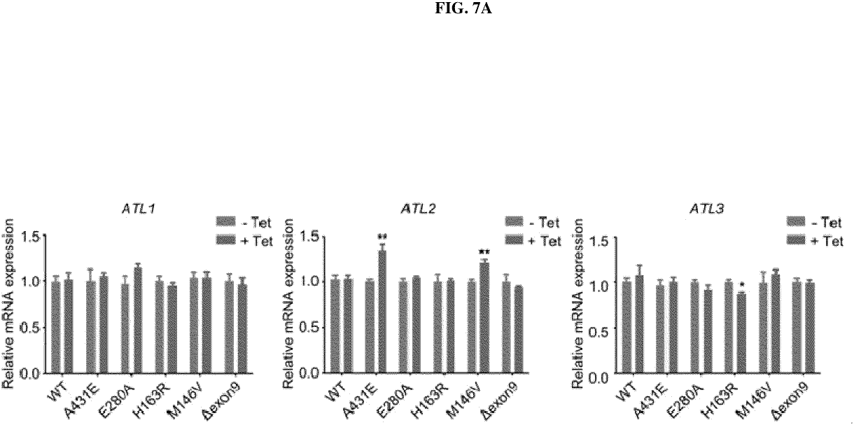

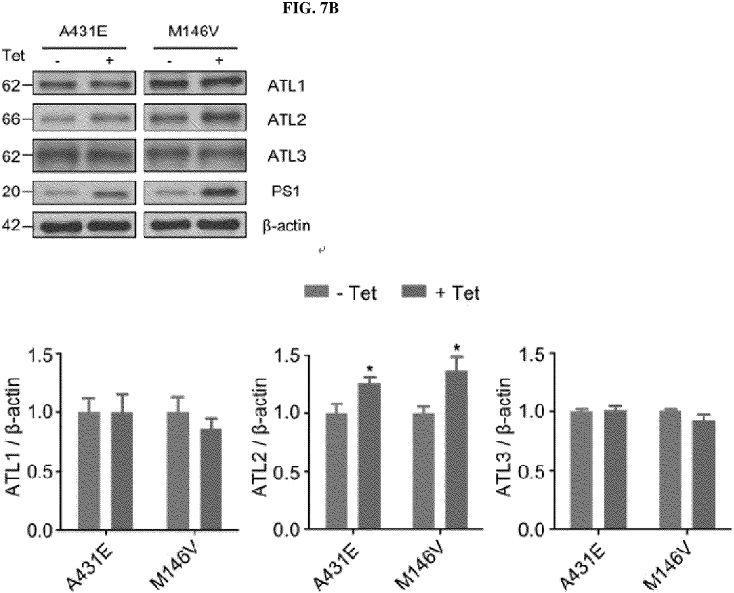

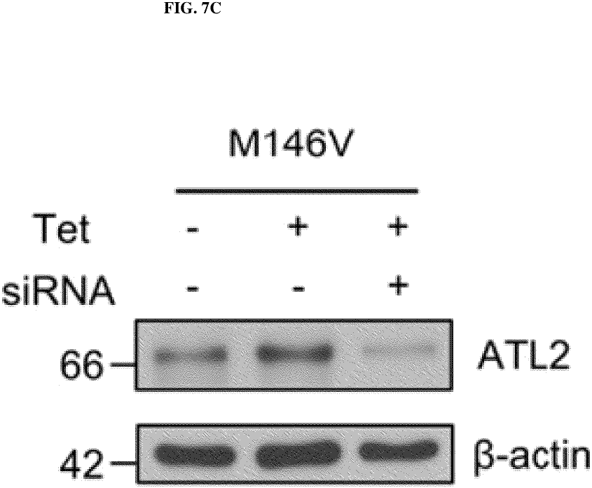

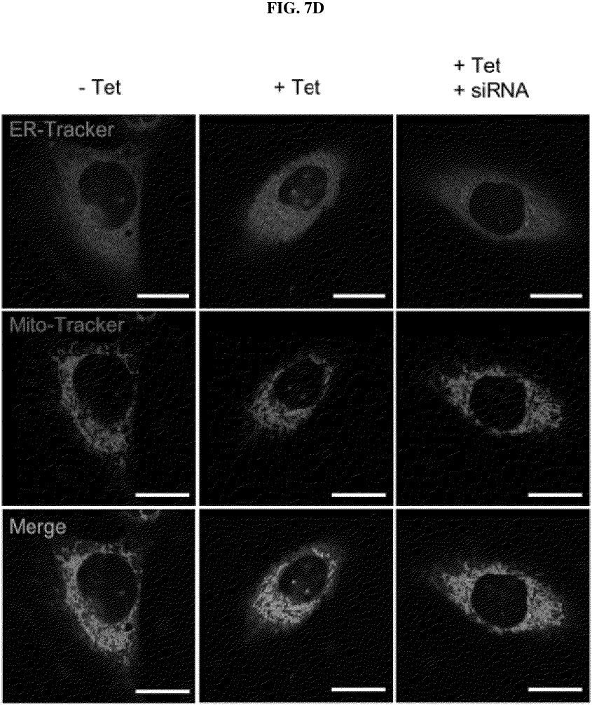

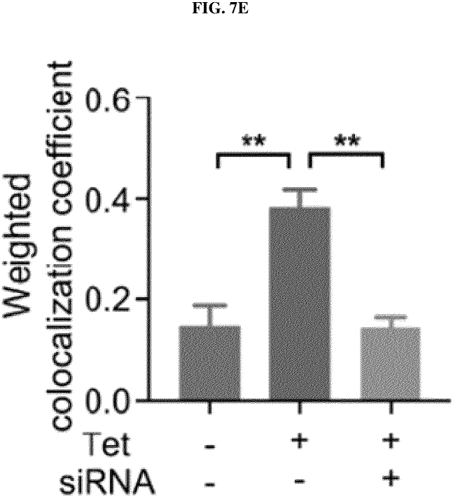

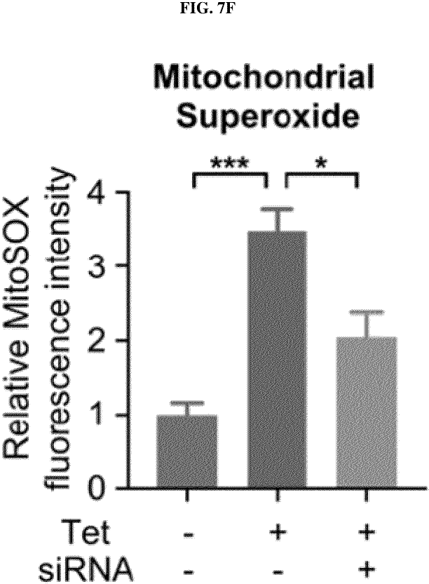

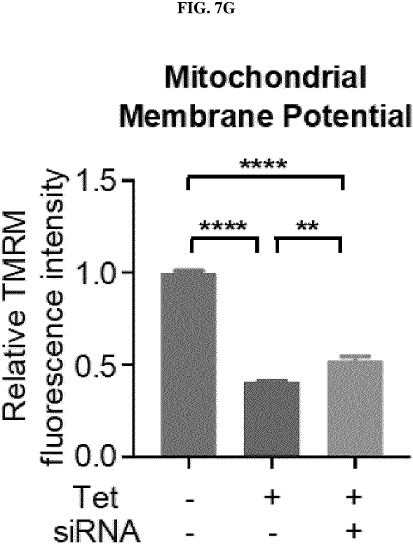

FIG. 7A shows the results of measuring the mRNA expression levels of ATL1, ATL2, and ATL3 in the H4^{PS1} cell lines before and after treatment with tetracycline. As shown in FIG. 7A, there was no significant difference in the mRNA expression of the ATL1 and ATL3, but the mRNA expression of the ATL2 was significantly increased in the H4^{PS1A431E} and H4^{PS}^{1M146V} cell lines after overexpressed PS1 mutants were induced.

FIG. 7B shows the results of performing the western blot on ATL1, ATL2, and ATL3 in the H4^{PS1A431E} and H4^{PS1M146V} cell lines before and after treatment with tetracycline. As shown in FIG. 7B, the expression of ATL2 protein was significantly increased in the H4^{PS1A431E} and H4^{PS1M146V} cell lines after the PS1 mutants were induced, consistent with the qPCR data, but there was no change in the expression of ATL1 and ATL3 proteins.

Since the ATL2 is involved in endoplasmic reticulum membrane fusion and tethering, which are important for forming the contacts between endoplasmic reticulum and mitochondria, it was hypothesized that the ATL2 may play an important role in the interactions between endoplasmic reticulum and mitochondria by physically increasing the tethering between endoplasmic reticulum and mitochondria. Indeed, previous studies have confirmed that the ATL2 is a protein involved in the contact between endoplasmic reticulum and mitochondria. To test this hypothesis, it was examined whether elevated colocalization of endoplasmic reticulum and mitochondria in the H4^{PS1M146V} cell line returned to its normal level as a result of knocking down the ATL2 after the PS1 mutants were induced.

FIG. 7C shows the results of carrying out the western blot on the ATL2 in the H4^{PS1M146V} cell line before and after treatment with tetracycline or transfection with siRNA, and, as shown in FIG. 7C, siRNA-mediated knockdown of the ATL2 was observed in the H4^{PS1M146V} cell line.

Then, endoplasmic reticulum-mitochondria colocalization was observed using a confocal microscope.

FIG. 7D shows the confocal images of the H4^{PS1M146V} cell lines labeled with Mito-Tracker (red) and ER-Tracker (green), which were taken before and after treatment with tetracycline or transfection with siRNA (Scale bar=20 µm), and FIG. 7E shows weighted colocalization coefficients between mitochondria and endoplasmic reticulum. 10 to 15 cells were analyzed for each imaging (n=3, **P<0.01). As shown in FIGS. 7D and 7E, endoplasmic reticulum-mitochondria colocalization was significantly elevated by inducing PS1M146V, and the elevated endoplasmic reticulum-mitochondria colocalization returned to its normal level upon knocking down the ATL2.

In addition, it was examined whether some of the mitochondrial dysfunction observed in the H4^{PS1M146V} cell line when overexpressed PS1 mutants were induced could be suppressed by knocking down the ATL2.

FIG. 7F shows the results of quantifying the production of mitochondrial superoxide in the H4^{PS1M146V} cell line before and after treatment with tetracycline or transfection with siRNA (n=4, *P<0.05, *** <0.001), and, as shown in FIG. 7F, it was found that the abnormally elevated level of the mitochondrial superoxide in the induced H4^{PS1M146V} cell line was reduced when the ATL2 was knocked down.

FIG. 7G shows the results of quantifying mitochondrial membrane potential in the H4^{PS1M146V} cell line with a TMRM probe before and after treatment with tetracycline or transfection with siRNA (n=4, **P<0.01, ****P<0.0001), and, as shown in FIG. 7G, it was observed that the damaged mitochondrial membrane potential was significantly elevated in the induced H4^{PS1M146V} cell line when the ATL2 was knocked down although it did not return to its normal level.

### Experimental Example 8. Elevation of the Expression Level of ATL2 In the Brains of Alzheimer’s Disease Mouse Models and Patients

The expression level of the ATL2 in the brains of mouse models and patients with Alzheimer’s disease was observed. Since the expression level of the ATL2 was elevated in the hippocampus of PS1M146V knock-in mice and the H4^{PS1M146V} cell line when overexpressed PS1 mutants were induced, the expression level of the ATL2 in the brains of 3xTg-AD mice with PS1M146V mutants was examined.

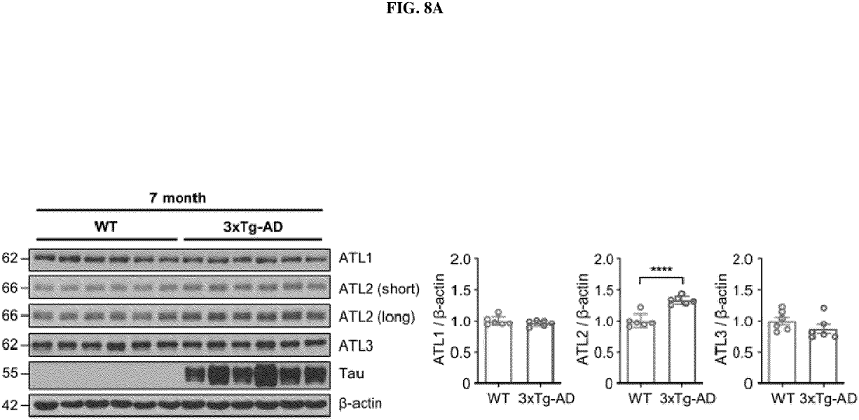

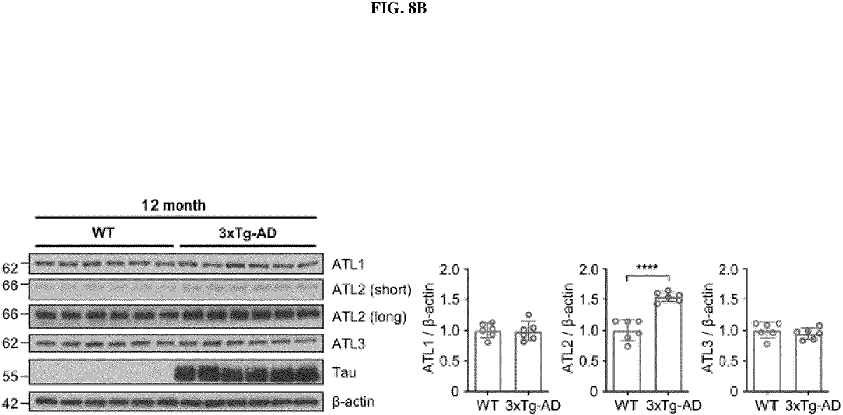

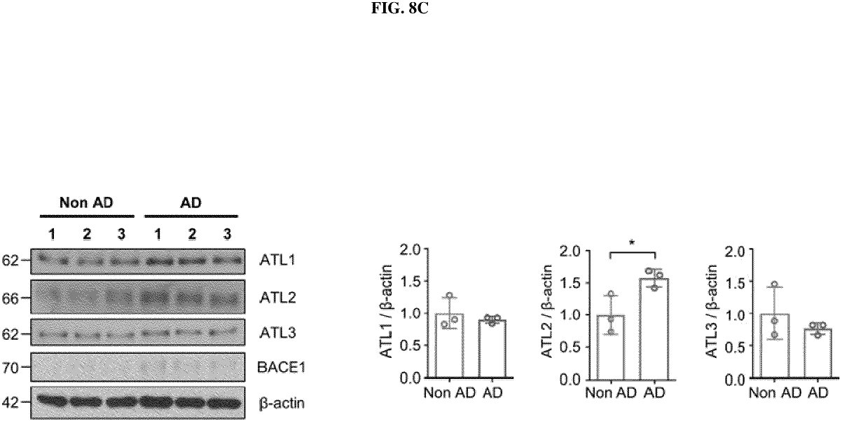

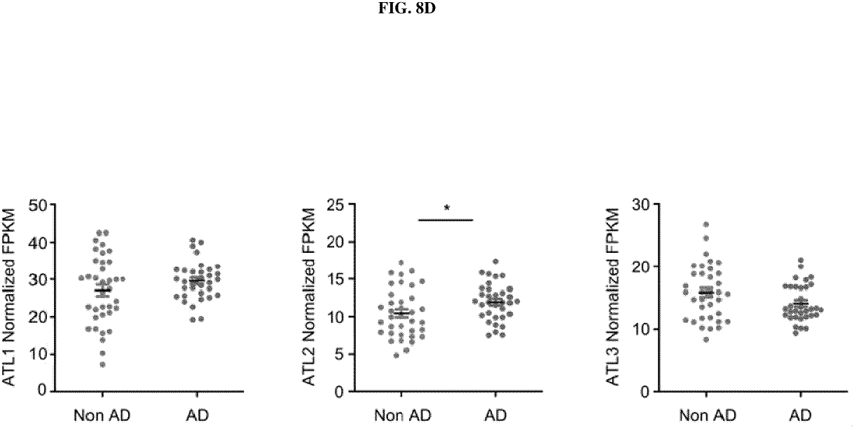

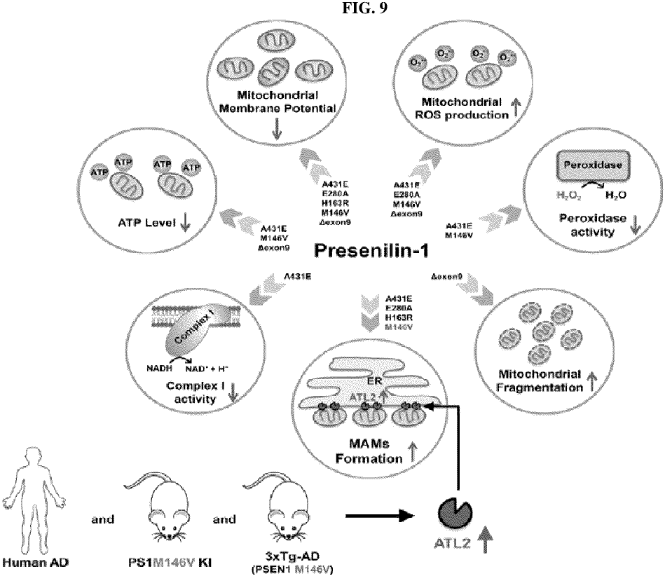

FIG. 8A shows the results of the western blot on ATL1, ATL2, ATL3, and Tau proteins in the hippocampus of seven-month-old 3xTg-AD mice and wild-type mice (two males and four females, respectively) (****P<0.0001), and FIG. 8B shows the results of the western blot on the ATL1, ATL2, ATL3, and Tau proteins in the hippocampus of 12-month-old 3xTg-AD mice and wild-type mice (two males and four females, respectively) (****P<0.0001).

As shown in FIGS. 8A and 8B, the ATL2 was significantly increased in the hippocampus of the 7- and 12-month-old 3xTg-AD mice compared to the wild-type mice of the same age, whereas there was no significant difference in the expression of the ATL1 and ATL3.

Next, it was examined whether the expression level of the ATL2 of the patients with Alzheimer’s disease was also elevated. The expression level of the ATL2 in rapidly harvested specimens from the inferior parietal lobule of the Alzheimer’s disease patients and age-matched controls was measured. The age, gender, post-mortem interval (PMI), and number of amyloid plaques are shown in Table 4 below.

TABLE 4

| Patients | Age (years) | Gender | PMI (h) | Neuritic plaques | Cause of Death | Braak stage |
|---|---|---|---|---|---|---|
| Control | | | | | | |
| 1 | 86 | Female | 2.25 | 7.6 | Unknown | 2 |
| 2 | 91 | Female | 4.00 | 10.4 | Unknown | 1 |
| 3 | 86 | Female | 3.75 | 7.8 | Cariovascular disease | 1 |
| Mean ± S.D. | 87.7 ± 2.9 | | 3.33 ± 0.95 | 8.6 ± 1.6 | | |

Alzheimer’s disease

| | | | | | | |
|---|---|---|---|---|---|---|
| 1 | 86 | Female | 4.25 | 23.4 | Bowel obstruction | 6 |
| 2 | 75 | Female | 2.33 | 19.0 | Congestive heart failure | 6 |
| 3 | 84 | Male | 4.50 | 34.8 | Unknown | 6 |
| Mean ± S.D. | 81.7 ± 5.9 | | 3.69 ± 1.19 | 25.7 ± 8.2 | | |

FIG. 8C shows the results of the western blot on ATL1, ATL2, ATL3, and BACE1 in the inferior parietal lobule of Alzheimer’s disease patients (n=3) and age-matched controls (n= 3). As shown in FIG. 8C, the expression level of the ATL2 of the AD patients was significantly elevated, whereas there was no significant difference in the expression levels of the ATL1 and ATL3.

In addition, the data published by the Allen Institute for Brain Science (Aging, Dementia, and TBI Study) was used to examine the expression level of the ATL2 gene in the brains of the patients with Alzheimer’s disease. The clinical data used for this research was from 107 subjects, of whom 57 were free of dementia and 50 were clinically diagnosed with dementia. Among the 57 dementia-free subjects, subjects with any of the following conditions were excluded: (1) Braak stage 6, (2) NIA-Reagan stage 3, (3) three or more traumatic brain injuries (TBI). As a result, 36 subjects (without Alzheimer’s disease) were selected. Of the 50 patients clinically diagnosed with dementia, 43 met the NINDS-ARDA’s criteria for diagnosing Alzheimer’s disease, either “pretty likely to have AD” or “likely to have AD.” Of these 43 Alzheimer’s disease subjects, subjects with any of the following conditions were excluded: (1) Braak stage 0, (2) NIA-Reagan stage 0, (3) three or more TBIs. As a result, 33 subjects (with Alzheimer’s disease) were selected.

Then, a set of RNA-seq data was analyzed to determine whether the expression of the ATL2 genes of the Alzheimer’s disease subjects was elevated.

FIG. 8D shows the expression levels of the ATL1, ATL2, and ATL3 genes in the frontal lobe white matter (FWM) of Alzheimer’s disease (red, n=33) or non-Alzheimer’s disease (blue, n=36) subjects. As shown in FIG. 8D, it was observed that the expression level of the ATL2 was significantly elevated in the frontal lobe white matter of the subjects with Alzheimer’s disease, but no significant difference was observed in the expression level of the ATL1 or ATL3.

In the present invention, it was found that PS1 mutants may result in mitochondrial dysfunction, such as increased binding between endoplasmic reticulum and mitochondria, increased mitochondrial ROS production, decreased mitochondrial membrane potential, decreased ATP production, decreased complex I activity, and decreased peroxidase activity, in brain glioma cells and that the PS1 mutants may abnormally increase the binding between endoplasmic reticulum and mitochondria by elevating the expression of the ATL2 in the brain. In addition, when the ATL2 was knocked down, it was observed that the binding between endoplasmic reticulum and mitochondria was lowered and that the expression of the ATL2 was elevated in the brains of Alzheimer’s disease animal models and patients. Accordingly, it is expected that it may possible to effectively prevent or treat Alzheimer’s disease by inhibiting the expression or activity of the ATL2 and that it may possible to diagnose the disease, predict the risk of developing the disease, and screen therapeutic agents for the disease, by measuring the level of the expression or activity of the ATL2.

The description of the present invention above is intended to provide its examples, and it is to be understood that the present invention can be easily modified into other specific forms without changing its technology or essential features by a person having ordinary skill in the technical field to which the present invention belongs. Therefore, it should be understood that the embodiments described above are illustrative in all respects and not restrictive.

What is claimed is:

1. A method of treating or alleviating Alzheimer’s disease, comprising administering the composition comprising, as an active ingredient, the inhibitor of the expression or activity of the Atlastin 2 (ATL2) gene or protein to a subject in need thereof.

2. The method of claim 1, wherein the ATL2 protein comprises an amino acid sequence of SEQ ID NO: 1.

3. The method of claim 1, wherein the ATL2 gene comprises a base sequence encoding the amino acid sequence of SEQ ID NO: 1.

4. The method of claim 1, wherein the inhibitor of the expression or activity of the ATL2 gene is one or more selected from the group consisting of microRNA (miRNA), small interference RNA (siRNA), short hairpin RNA (shRNA), peptide nucleic acid (PNA), antisense nucleotides, and ribozymes, which specifically bind to the mRNA of the ATL2 gene.

5. The method of claim 1, wherein the inhibitor of the expression or activity of the ATL2 protein is one or more selected from the group consisting of compounds, peptides, peptide mimics, substrate analogs, aptamers, and antibodies, which specifically bind to the ATL2 protein.

6. The method of claim 1, wherein the inhibitor of the expression or activity of the ATL2 gene or protein lowers the binding between endoplasmic reticulum (ER) and mitochondria in the brain.

7. The method of claim 1, wherein the inhibitor of the expression or activity of the ATL2 gene or protein inhibits the production of mitochondrial superoxide.

8. A method of diagnosing Alzheimer’s disease, comprising measuring the level of the expression or activity of Atlastin 2 (ATL2) gene or protein in a sample collected from the brain of a subject.

9. The method of claim 8, wherein the level of the expression or activity of the ATL2 gene is measured using one or more selected from the group consisting of primers, probes, and antisense nucleotides, which specifically bind to the mRNA of the ATL2 gene.

10. The method of claim 8, wherein the level of the expression or activity of the ATL2 protein is measured using antibodies or aptamers that specifically bind to the ATL2 protein.

11. The method of claim 8, wherein the ATL2 protein comprises an amino acid sequence of SEQ ID NO: 1.

12. The method of claim 8, wherein the ATL2 gene comprises a base sequence encoding the amino acid sequence of SEQ ID NO: 1.

13. The method of claim 8, wherein the method further comprises diagnosing a subject with Alzheimer’s disease when the level of the expression or activity of the ATL2 gene or protein is higher than that of a normal control group.

14. A method of predicting the risk of developing Alzheimer’s disease, comprising measuring the level of the expression or activity of Atlastin 2 (ATL2) gene or protein in a sample collected from the brain of a subject.

15. The method of claim 14, wherein the method further comprises predicting a high risk of developing Alzheimer’s disease when the level of the expression or activity of the ATL2 gene or protein is higher than that of a normal control group.

16. A method of screening candidate substances for treatment of Alzheimer’s disease, comprising:
treating overexpressed presenilin-1 (PS1) cells collected from a subject having Alzheimer’s disease with a candidate substance;
measuring the level of the expression or activity of Atlastin 2 (ATL2) gene or protein in the cells; and
selecting a candidate substance as a candidate substance for treatment of Alzheimer’s disease when the level of the expression or activity of the ATL2 gene or protein is lower than the level measured before the cells were treated with the candidate substance.


SEQUENCE LISTING (12 sequences)

SEQ ID NO: 1 - protein, 579 aa, synthetic construct
  REGION 1..579: note = ATL2
MAEGDEAARGQQPHQGLWRRRRTSDPSAAVNHVSSTTSLGENYEDDDLVNSDEVMKKPCPVQIVLAHEDDHNFELDEEALEQILLQEHIRDLNIVVVSVAGAFRKGKSFLLDFMLRYMYNKDSQSWIGGNNEPLTGFTWRGGCERETTGIQVWNEVFVIDRPNGTKVAVLLMDTQGAFDSQSTIKDCATVFALSTMTSSVQVYNLSQNIQEDDLQHLQLFTEYGRLAMEEIYQKPFQTLMFLIRDWSYPYEHSYGLEGGKQFLEKRLQVKQNQHEELQNVRKHIHNCFSNLGCFLLPHPGLKVATNPSFDGRLKDIDEDFKRELRNLVPLLLAPENLVEKEISGSKVTCRDLVEYFKAYIKIYQGEELPHPKSMLQATAEANNLAAVAGARDTYCKSMEQVCGGDKPYIAPSDLERKHLDLKEVAIKQFRSVKKMGGDEFCRRYQDQLEAEIEETYANFIKHNDGKNIFYAARTPATLFAVMFAMYIISGLTGFIGLNSIAVLCNLVMGLALIFLCTWAYVKYSGEFREIGTVIDQIAETLWEQVFSKLFEVTRRRMVHRALSSAQRQRLSSNNNKKKN

SEQ ID NO: 2 - DNA, 4282 bp, synthetic construct
  misc_feature 1..4282: note = ATL2
  source 1..4282: mol_type = other DNA
aaatcggtacaagatggcggagggggacgaggcagcgcgagggcagcaaccgcaccaggggctgtggcgccggcgacggaccagcgacccaagcgccgcggttaaccacgtctcgtccacgacctccctaggtgagaattatgaagatgatgacctagtaaattctgatgaggttatgaagaaaccatgtccagtacagattgttcttgctcatgaagatgaccataactttgaacttgatgaagaagctttggagcagatattgctacaggagcacatacgagatcttaacatagtagtggtatctgtggcaggagcttttcgtaaagggaagtcatttctactggacttcatgcttagatacatgtataacaaggattctcaaagttggattggtggaaacaatgaaccattgacaggctttacatggcgaggtggctgtgaaagagaaacaacaggcatacaagtttggaatgaagtatttgtgattgacagacctaatggaactaaagttgctgtgctgcttatggatacccagggtgcctttgatagccagtcaactatcaaagactgtgcaacggtgtttgctctgagcactatgactagctctgtccaggtatataatctgtctcagaatattcaagaagatgatcttcaacatttgcaattatttacagagtatggaagacttgcgatggaagaaatctaccagaaaccatttcagacattaatgtttttgattcgagattggagctatccttatgaacattcatatggtttggaaggtggaaagcaatttcttgaaaagagattacaggtaaaacaaaatcaacatgaagagcttcagaatgtaaggaagcacatacacaattgtttctcaaatcttggttgcttccttttgccacatcctggtcttaaagttgcaactaatcctagttttgatgggagattgaaagatattgatgaagactttaaacgagagcttcgaaatctggttccattgctgcttgcccctgaaaatttggtagaaaaagagataagtggatctaaagtcacttgtagagatcttgtagaatattttaaggcttacatcaaaatctatcaaggagaagaacttccacatccaaagtccatgcttcaggcaacagctgaagctaataatcttgctgcagtagcaggagcaagagatacctattgtaaaagtatggaacaggtatgtggaggggacaagccttacattgcaccttcagatctggagcgaaaacacttggatctcaaggaagtggcgataaaacaatttcgttcagtaaaaaagatgggtggagatgagttctgccgtcgttatcaggaccagcttgaagctgaaattgaagaaacctatgcaaattttataaagcacaatgatggcaaaaatatcttctatgctgctcgtaccccagccacactgtttgcggtcatgtttgctatgtatataatctcaggactgactggcttcattggcctaaactctatagctgtcttgtgtaaccttgtcatggggttagcactgatatttctttgtacttgggcatatgttaaatactctggggagttcagagaaattggaacagtgattgatcagattgctgaaacactatgggaacaggtgttttccaaactgtttgaagttactagacgtcgaatggttcaccgtgctctttcatcagcacagcgacagagactgtcatccaacaataacaagaagaaaaattagacagtatttttaacctttttctctatctgaagtgttcacacttacacatgtaggacaataagcaggaccgtctgggccggtctgcataaatgctgtatacataccagatttgatgctgcatatagggtatggaattgcacatccatctcataggaattgtaaatggtttgaataagaggaaagtaatttttgttgcattataaaatgtctagtagcatcataagtttttttgagagaagcatctttttatttcccatattcctggttattttcatcattgctttgaattgaatttttatatctatttttatatgtaactctttttttacctcatgtttttgtttgttttgcacatttctcataccacaggtattgaagcccctgggtgataatttgatggaggaaaacataaggcagtctgtaacaaactctatcaaagcaggcctgactgaccaggtgtctcatcatgccagattaaagacagactgacagttcatctcctcacggactccactctctttttttttcatgcttgctgtacaatgagaactcaaataaaaataaaccaaagtttacaatcaactgtagaagtagtttagtgtaactggcttcacagatggctgccacagagtgtgaagattgtttgttagttttaagcattcttttaatggctcctaagacatgcagatggactgaggagcattggttaatcatgcacctttgtgccatgtttaactcttttatttctttttacttaatctaatgttagtgaatttgtcttatgtaaaaggatatttcagggaaatattttcagaaatctatttagagtctctttaacacagtgtcccattgaaattttaatttttagagaatttatgaatcactgtttcaagaaccagattggaaagacaatgaagcctttattgagccactacattaaaagtatatattgctttactgccttcaataccagtattacatcaatgcatgtatcagaaacttcacagaaattacatggcaactcttgtagctaagaaagtaattctgaggtgtacatttgtcttgcctttttaaatttataaacttgccctaaaaggagatgcatatctgggaaactgaactgtctttttgcagtttagccttcatgtatataaaatatgccattaattttattggggaagaaattccatccaaaaatgttgcctacagctatgagttaagagtgtctgtacagtgtgtagcttttattttctaaaatcacagatagggcatgtatatgacttataaatatataaatacgattttgtattaaaagttttgtagtttatggcaaaatctggtcctgtggtaggctaaataagtactgtccctgtgaaaggaatgtttgtggctcatgtcagtgtgtgaatgcatagacaatttgaagtttttgatatatttgtgatatttatcttgagcactgcaatctcaccccccccccgcccaccaagggaattcaatgggaatgtttattgtgactttgtcctctgttgcattttaaagttatttcctgtaatttattttcagtacataattaaaaatttgttgtatatataaaatgaaacttgtgattcttttttaaggaatttttcaagtatgtattccacttcataaaccacatttgtttatagtgaaaagagcatgagcaggaaacaccccagttgagagctctaaggcattgtacctcaaaactaagcttgtaaaaagttacttttctgtgtctgtctctataagggaaaacctatgattagcctagttgtagatggggattgttaatttctttctctctctttttttttttttggtggtatatgtaaggctagcattttttcagtacttttgtggtcttctttgtgaacaccgattaaagtacctagttttgtttgtttcttttttgagacggagtctctctctgtcacccaggctggagtgcagtggtgcgatcttggctcactgcaacctccacctcctgggttcaagtgattctcccgcctcagcctcctgagtagctgggactacaggtgcgtgccaccattcccggctaatgttttctattttaagtagagatggggtttcaccgttttagccaggatggtctcaatcgcctgaccttgtgatccgcctgccttggcctcccaaagtgttgggattacaggcgtgagccaccgcgcccggcccttagtttgctttttaagcgaagagaatgcaggtgttgagttgcatgtgtgaaaactctgaatttactttggttgatacatcttcgctcccactcccattcagattctgtttttatatggattgtatttgttttcatctgtattaaattgctaacttatctgttcttaaagaatccacctgaaaagtgtctttttatttcttctgccaccctgttttatgaatgctgtggactgttttttatgaatgaaaaaataatttgttgtataatagcagattttctgaaattaaagacagcttacttttaaaaagtta

SEQ ID NO: 3 - RNA, 19 nt, synthetic construct
  misc_feature 1..19: note = ATL2 sense
  source 1..19: mol_type = other RNA
tagagtttgttacagactg

SEQ ID NO: 4 - RNA, 19 nt, synthetic construct
  misc_feature 1..19: note = ATL2 antisense
  source 1..19: mol_type = other RNA
cagtctgtaacaaactcta

SEQ ID NO: 5 - DNA, 20 bp, synthetic construct
  misc_feature 1..20: note = ATL1 forward
  source 1..20: mol_type = other DNA
ccctgtgcacttgggcatat

SEQ ID NO: 6 - DNA, 21 bp, synthetic construct
  misc_feature 1..21: note = ATL1 reverse
  source 1..21: mol_type = other DNA
ttgtacaaagcctggtcccac

SEQ ID NO: 7 - DNA, 21 bp, synthetic construct
  misc_feature 1..21: note = ATL2 forward
  source 1..21: mol_type = other DNA
ttgccacatcctggtcttaaa

SEQ ID NO: 8 - DNA, 20 bp, synthetic construct
  misc_feature 1..20: note = ATL2 reverse
  source 1..20: mol_type = other DNA
gcagcaatggaaccagattt

SEQ ID NO: 9 - DNA, 20 bp, synthetic construct
  misc_feature 1..20: note = ATL3 forward
  source 1..20: mol_type = other DNA
acaagccctgactttgatgg

SEQ ID NO: 10 - DNA, 20 bp, synthetic construct
  misc_feature 1..20: note = ATL3 reverse
  source 1..20: mol_type = other DNA
tgcagctgctaagttgttgg

SEQ ID NO: 11 - DNA, 19 bp, synthetic construct
  misc_feature 1..19: note = GAPDH forward
  source 1..19: mol_type = other DNA
agccacatcgctcagacac

SEQ ID NO: 12 - DNA, 19 bp, synthetic construct
  misc_feature 1..19: note = GAPDH reverse
  source 1..19: mol_type = other DNA
gcccaatacgaccaaatcc